(19) United States
(12) Patent Application Publication
(10) Pub. No.: US 2009/0036321 A1
(43) Pub. Date: Feb. 5, 2009

(54) Methods for predicting the course of a malignant disease

(75) Inventors: Edward Moler, Walnut Creek, CA (US); Filippo Randazzo, Mercer Island, WA (US); Michael Rowe, Oakland, CA (US)

Correspondence Address: FOLEY AND LARDNER LLP;SUITE 500, 3000 K STREET NW, WASHINGTON, DC, 20007, US

(73) Assignee: Chiron Corporation, EMERYVILLE (CA)

(21) Appl. No.: 10/559,480

(22) PCT Filed: Jun. 3, 2004
(86) PCT No.: PCT/US04/17408

§ 371 Date: Oct. 11, 2006

Related U.S. Application Data
(60) Provisional application No. 60/475,872, filed on Jun. 3, 2003.

(51) Int. Cl. C40B 30/04; C12Q 1/02; G01N 33/574; C12Q 1/68

(52) U.S. Cl. 506/9; 435/29; 435/6; 435/7.23

(57) ABSTRACT

The present invention relates to methods of predicting the course of malignant disease and more specifically to methods which use SERPINE2 as a prognostic indicator of disease in cancer patients.

### CROSS-REFERENCE TO RELATED PATENT APPLICATIONS

This application claims benefit of priority to U.S. Provisional Application No. 60/475,872, filed Jun. 3, 2003 and incorporated herein by reference.

### FIELD OF THE INVENTION

The present invention relates to methods of predicting the course of malignant disease and more specifically to methods that use SERPINE2 as a prognostic indicator of disease in cancer patients.

### BACKGROUND OF THE INVENTION

Cancer is a major cause of morbidity of adults in the United States. The National Center for Chronic Disease Prevention and Health Promotion states that cancer was the second leading cause of death in the United States in 2000. See Miniño et al. NATIONAL VITAL STATISTICS REPORTS; 50(15):1-120 (2002). Unfortunately, many cancer patients are asymptomatic until very late in the course of disease. Such late diagnosis delays treatment of patients until tumors become highly invasive and metastatic. Treatment then becomes difficult, and there is a low rate of curative resections and high rate of relapse. Thus, accurate, early prognosis of asymptomatic cancers is an important tool to guide modalities of treatment.

Examples of malignant diseases needing early prognosis and tailored modalities of treatment include colon and rectal cancers. Colon and rectal cancers are malignant diseases that occur in different segments of the large intestine. In many respects, both are considered the same disease, and are sometimes referred to as “colorectal cancer” (CRC). The major difference between these two diseases is the site at which the malignant tumors, sometimes referred to as polyps, occur and the modalities of treatment of the disease. Treatment modalities differ as required by the location of the malignant tumors. CRC is the second leading cause of cancer-related death in the United States, and the third most common cancer in men and women. See Centers for Disease Control and Prevention. Colorectal Cancer: The Importance of Prevention and Early Detection (2003). Reducing the number of deaths from CRC depends both on the prophylactic detection and removal of precancerous colorectal polyps and on the early detection and treatment of malignant disease and cancerous polyps. Although precancerous polyps may persist for years before malignant disease begins, CRC may be prevented by removing precancerous polyps or other growths.

To detect these polyps and growths, the Centers for Disease Control and Prevention (CDC) recommends four screening techniques for CRC. First, the CDC recommends a fecal occult blood test to detect minute traces of blood in a stool sample. Second, the CDC recommends physicians conduct flexible sigmoidoscopy exams in order to visually inspect the interior walls of the rectum and some parts of the colon. Third, the CDC recommends physicians conduct colonoscopy exams to visually inspect the interior walls of the rectum and the entire colon. Lastly, a patient may undergo a double-contrast barium enema test in order to allow the X-ray visualization of the rectum and colon. In addition, a digital rectal exam may be used to detect some forms of CRC, but this procedure is of limited use as it allows for inspection of only a limited area. Because CRC are generally asymptomatic until very late in the disease course, these screening techniques play a key role in strategies to combat CRC. However, perhaps because of the invasiveness and cost of these techniques, screening for CRC lags far behind screening for other cancers.

In addition, targeting treatment modalities for late-stage CRC is difficult. Currently, the prognosis of patients with CRC is related to three characteristics: the degree of penetration of the tumor through the bowel wall, the presence or absence of nodal involvement, and the presence or absence of distant metastases. These three characteristics form the basis of all staging systems developed for CRC, and each depends upon the professional judgment of the attending physician. Additionally, bowel obstruction and bowel perforation, see Steinberg et al. CANCER 57(9): 1866-70 (1986), and elevated pretreatment serum levels of carcinoembryonic antigen, see Filella et al. ANN. SURG. 216(1): 55-9 (1992), have a negative prognostic significance. Treatment decisions depend on the stage of the disease and physician and patient preferences rather than the age of the patient. See Fitzgerald et al. DIS. COLON RECTUM. 36(2): 161-6 (1993); Chiara et al. CANCER CHEMOTHER. PHARMACOL. 42(4): 336-40 (1998)); and Popescu et al. J. CLIN. ONCOL. 17(8): 2412-8 (1999). CRC is highly treatable when localized to the bowel, and the primary treatment modality is surgical resection, either through open colectomy or laparoscopic-assisted colectomy. Adjuvant chemotherapy is also available for CRC patients but the potential value for those in some disease stages remains controversial. While combined modality therapy with chemotherapy and radiation therapy has a significant role in the management of patients with rectal cancer, the role of adjuvant radiation therapy for patients with colon cancer has not been well defined. In addition, treatment modalities for patients with recurrent or advanced colon cancer depend on the location of the disease. For patients with locally recurrent or some forms of metastatic disease, surgical resection, if feasible, is the only potentially curative treatment. Patients with unresectable disease are treated with systemic chemotherapy. Disease recurrence following surgery is a major problem and is often the ultimate cause of death. However, studies attempting to define “high-risk” groups of CRC have closed early due to analysis of patient data which demonstrated no benefit for the group receiving radiation therapy with respect to relapse or overall survival. See Martenson et al. PROC. AM. SOC. CLIN. ONCOL. 18: A-904, 235a (1990).

Therefore, a strong need still exists for method of predicting the course of malignant disease in individuals. Ideally, a prognosis will be based on cellular indicators that correlate strongly with disease outcome.

### SUMMARY OF THE INVENTION

The present invention relates to methods of predicting the course of malignant disease in an individual. In particular embodiments, the malignant disease is adult soft tissue carcinoma, colorectal carcinoma, hepatocellular carcinoma, or pancreatic carcinoma. The invention also relates to the prognosis or diagnosis of relapse of a malignant disease after treatment.

In one embodiment, the methods comprise (a) determining the level of expression of a SERPINE2 gene product within a patient biological sample, (b) comparing the level to a control level, and (c) correlating the comparison of step (b) to a predicted course of malignant disease. The control level may be derived from a predetermined standard, an immortalized cell tissue culture, a primary cell tissue culture or a non-malignant tissue from the same patient. SERPINE2 gene product levels in a metastatic tumor and a primary tumor from the same individual also may be compared.

In another embodiment of the invention, differential expression of SERPINE2 is utilized as one of a battery of prognostic indicators.

In another aspect, the invention relates to the measurement of nucleotides or proteins to determine the differential expression of SERPINE2. This aspect of the invention encompasses a wide range of techniques known in the art that are useful for determining a quantitative or qualitative difference in expression of SERPINE2, including northern, Southern, and western blots; polymerase chain reaction (PCR) and related competitive and quantitative testing; and cDNA and protein microarrays. Another aspect of the invention may utilize antibodies to measure differential expression of SERPINE2. The invention also provides kits utilizing these techniques to measure the differential expression of SERPINE2.

Finally, the invention also provides methods for identification of SERPINE2 binding partners and methods of using SERPINE2 binding partners as prognostic indicators of disease, or as useful to the measurement of differential expression of SERPINE2.

### BRIEF DESCRIPTION OF THE DRAWINGS

FIG. 1 depicts one nucleotide sequence of Homo sapiens serine (or cysteine) proteinase inhibitor, clade E, member 2 (SERPINE2), mRNA. This sequence is designated as GENEBANK Locus NM_{—}006216.1.

FIG. 2 depicts one nucleotide sequence of Homo sapiens serine (or cysteine) proteinase inhibitor, clade E, member 2 (SERPINE2), mRNA. This sequence is designated as GENEBANK Locus NM_{—}006216.2.

FIG. 3 depicts the amino acid sequence of Homo sapiens serine (or cysteine) proteinase inhibitor, clade E, member 2 (SERPINE2). SERPINE2 is also known as plasminogen activator inhibitor type 1, member 2; protease inhibitor 7 (protease nexin I); glial-derived nexin 1; glial-derived neurite promoting factor. This sequence is designated as NCBI Entrez Protein Locus NP_{—}006207.

FIG. 4 is a chart depicting a biological association network of 21 genes identified from Table 1. This figure assists in visualizing and identifying the direct interactions between genes. Abbreviations for the identified genes are listed in Table 1.

FIG. 5 is a graph demonstrating the correlation between CRC patient survival and expression of SERPINE2 in patient biological samples. Survival, as the natural logarithm of days plus one from sample collection to the date that the patient was known to be alive, was plotted against the logarithmically transformed expression ratio of SERPINE2 in primary colon tumor relative to adjacent normal colon epithelium.

FIG. 6 is a graph demonstrating the tendency of patients expressing lower levels of SERPINE2 in the colon tumors relative to the nearby colon epithelium to outlive patients with higher levels of SERPINE2. The data are presented in a Kaplan-Meier analysis. The data shown in the left plot were generated using cDNA microarrays; the data in the right plot were generated using quantitative RT-PCR. The patients have been divided into two groups on the basis of SERPINE2 expression in the primary colon tumor relative to normal epithelium. For each group, the fraction of patients surviving as a function of time is indicated by one of the lines. The Gehan statistic indicates that the overall survival for those patients with lower levels of SERPINE2 expression is significantly greater with a p-value of <0.05 using either method.

### DETAILED DESCRIPTION OF THE PREFERRED EMBODIMENTS

The following description outlines the invention summarized above. The invention, however, is not limited to the particular methodology, protocols, cell lines, animal species or genera, constructs, and reagents described and as such may vary. Likewise, the terminology used herein describes particular embodiments only, and is not intended to limit the scope of the invention.

Unless defined otherwise, all technical and scientific terms used herein have the same meaning as commonly understood to one of ordinary skill in the relevant art.

All publications and patents mentioned herein are incorporated herein by reference. Reference to a publication or patent, however, does not constitute an admission as to prior art.

### I. DEFINITIONS

For convenience, the meaning of certain terms and phrases employed in the specification, examples, and appended claims are provided below.

The singular forms “a,” “an,” and “the” include plural reference unless the context clearly dictates otherwise.

“Polynucleotide” and “nucleic acid,” used interchangeably herein, refer to polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids. Polynucleotides may further comprise genomic DNA, cDNA, or DNA-RNA hybrids.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical.” The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

Another way of viewing sequence identity in the context to two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Gene” refers to a polynucleotide sequence that comprises control and coding sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene.

“Expression” generally refers to the process by which a polynucleotide sequence undergoes successful transcription and translation such that detectable levels of the amino acid sequence or protein are expressed. In certain contexts herein, expression refers to the production of mRNA. In other contexts, expression refers to the production of protein.

“Differential expression” refers to both quantitative as well as qualitative differences in the temporal and tissue expression patterns of a gene. For example, a differentially expressed gene may have its expression activated or completely inactivated in normal versus disease conditions. Such a qualitatively regulated gene may exhibit an expression pattern within a given tissue or cell type that is detectable in either control or disease conditions, but is not detectable in both. “Differentially expressed polynucleotide” refers to a polynucleotide sequence that uniquely identifies a differentially expressed gene so that detection of the differentially expressed polynucleotide in a sample is correlated with the presence of a differentially expressed gene in a sample. “Differentially expressed protein” refers to an amino acid sequence that uniquely identifies a differentially expressed protein so that detection of the differentially expressed protein in a sample is correlated with the presence of a differentially expressed protein in a sample.

“Cancer,” “neoplasm,” “tumor,” and “carcinoma,” used interchangeably herein, refer to cells or tissues that exhibit an aberrant growth phenotype characterized by a significant loss of control of cell proliferation. The methods and compositions of this invention particularly apply to precancerous (i.e., benign), malignant, pre-metastatic, metastatic, and non-metastatic cells.

“Cell type” refers to a cell from a given source (e.g., tissue or organ) or a cell in a given state of differentiation, or a cell associated with a given pathology or genetic makeup.

The phrase “cells that express SERPINE2” refers to any cell that expresses detectable levels of SERPINE2. SERPINE2 protein may be detected using methods such as, but not limited to, quantitative reverse transcription polymerase chain reaction (qRT-PCR), enzyme-linked immunosorbent assay (ELISA), radioimmunoassay (RIA), microarray methods or immunoflourescence. An mRNA encoding SERPINE2 protein may be detected by Northern blots, polymerase chain reaction (PCR), microarray methods, or in situ hybridization. Other methods for detecting specific polynucleotides or polypeptides are discussed herein and are well known to those skilled in the art.

The phrase “cells that overexpress and/or upregulate SERPINE2” refers to cells wherein the SERPINE2 protein or mRNA transcript is expressed at higher levels than in corresponding normal cells. For example, in a cell that overexpresses and/or upregulates SERPINE2, the mRNA or protein may be produced at levels at least about 20% higher, at least about 25% higher, at least about 30% higher, at least about 35% higher, at least about 40% higher, at least about 45% higher, at least about 50% higher, at least about 55% higher, at least about 60% higher, at least about 65% higher, at least about 70% higher, at least about 75% higher, at least about 80% higher, at least about 85% higher, at least about 90% higher, at least about 95% higher, at least about 100% or more higher, at least about at least about 1.2-fold higher, at least about 1.5-fold higher, at least 1.75-fold higher, at least about 2-fold higher, at least about 5-fold higher, at least about 10-fold higher, or at least about 50-fold or more higher than that of a corresponding normal cell. In a specific embodiment, in a cell that overexpresses and/or upregulates SERPINE2, the SERPINE2 mRNA may be produced at levels at least about 1.5-fold higher than that of a corresponding normal cell. In another embodiment of the invention, SERPINE2 mRNA may be produced at levels at least about 1.75-fold higher than that of a corresponding normal cell. In a further embodiment, SERPINE2 mRNA may be produced at levels at least about 2.0-fold higher than that of a corresponding normal cell. The comparison may be made between different tissues or between different cells.

“Polypeptide” and “protein,” used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

An “expression product” or “gene product” is a biomolecule, such as a protein or mRNA, that is produced when a gene in an organism is transcribed or translated or post-translationally modified.

“Host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

“SERPINE2 binding partner” refers to a molecule that binds to SERPINE2 polypeptides or polynucleotides. In a specific embodiment of the invention, a SERPINE2 binding partner is a polypeptide, i.e., a polypeptide SERPINE2 binding partner. Examples of polypeptide SERPINE2 binding partners include, but are not limited to, immunoglobulins (antibodies), and functional equivalents thereof, peptides generated by rational design, etc. In another embodiment, a SERPINE2 binding partner may comprise a polynucleotide, i.e., a polynucleotide SERPINE2 binding partner. In yet another embodiment, a SERPINE2 binding partner may comprise a small molecule, i.e., a small molecule SERPINE2 binding partner.

In the context of SERPINE2, the term “functional equivalent” refers to a protein or polynucleotide molecule that possesses functional or structural characteristics that are substantially similar to all or part of the native SERPINE2 protein or native SERPINE2-encoding polynucleotides. A functional equivalent of a native SERPINE2 protein may contain modifications depending on the necessity of such modifications for a specific structure or the performance of a specific function. The term “functional equivalent” is intended to include the “fragments,” “mutants,” “derivatives,” “alleles,” “hybrids,” “variants,” “analogs,” or “chemical derivatives” of native SERPINE2.

In the context of immunoglobulins, the term “functional equivalent” refers to immunoglobulin molecules that exhibit immunological binding properties that are substantially similar to the parent immunoglobulin. “Immunological binding properties” refers to non-covalent interactions of the type that occurs between an immunoglobulin molecule and an antigen for which the immunoglobulin is specific. Indeed, a functional equivalent of a monoclonal antibody immunoglobulin, for example, may inhibit the binding of the parent monoclonal antibody to its antigen. A functional equivalent may comprise F(ab′)2 fragments, F(ab) molecules, Fv fragments, single chain fragment variable displayed on phage (scFv), single domain antibodies, chimeric antibodies, or the like so long as the immunoglobulin exhibits the characteristics of the parent immunoglobulin.

“Isolated” refers to a polynucleotide, a polypeptide, an immunoglobulin, or a host cell that is in an environment different from that in which the polynucleotide, the polypeptide, the immunoglobulin, or the host cell naturally occurs.

“Substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated.

“Diagnosis” and “diagnosing” generally includes a determination of a subject's susceptibility to a disease or disorder, a determination as to whether a subject is presently affected by a disease or disorder, a prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived therefrom and the progeny thereof. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components.

“Individual,” “subject,” “host,” and “patient,” used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Hybridization” refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

“Biomolecule” includes polynucleotides and polypeptides.

“Biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

“Oligonucleotide” refers to a polynucleotide sequence comprising, for example, from about 10 nucleotides (nt) to about 1000 nt. Oligonucleotides for use in the invention are preferably from about 15 nt to about 150 nt, more preferably from about 150 nt to about 1000 nt in length. The oligonucleotide may be a naturally occurring oligonucleotide or a synthetic oligonucleotide.

“Modified oligonucleotide” and “Modified polynucleotide” refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′,5′-3′, or 5′-5′ linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e.g., hexose phosphodiester; peptide nucleic acids). The modifications may be internal (single or repeated) or at the end(s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e.g., 3′-substituted ribonucleotides or deoxyribonucleotide monomers), any of which are bound together via 5′ to 3′ linkages.

“Biomolecular sequence” or “sequence” refers to all or a portion of a polynucleotide or polypeptide sequence.

The term “microarray” refers generally to the type of genes or proteins represented on a microarray by oligonucleotides (polynucleotide sequences) or protein-capture agents, and where the type of genes or proteins represented on the microarray is dependent on the intended purpose of the microarray (e.g., to monitor expression of human genes or proteins). The oligonucleotides or protein-capture agents on a given microarray may correspond to the same type, category, or group of genes or proteins. Genes or proteins may be considered to be of the same type if they share some common characteristics such as species of origin (e.g., human, mouse, rat); disease state (e.g., cancer); functions (e.g., protein kinases, tumor suppressors); same biological process (e.g., apoptosis, signal transduction, cell cycle regulation, proliferation, differentiation). For example, one microarray type may be a “cancer microarray” in which each of the microarray oligonucleotides or protein-capture agents correspond to a gene or protein associated with a cancer. An “epithelial microarray” may be a microarray of oligonucleotides or protein-capture agents corresponding to unique epithelial genes or proteins. Similarly, a “cell cycle microarray” may be a microarray type in which the oligonucleotides or protein-capture agents correspond to unique genes or proteins associated with the cell cycle.

The term “detectable” refers to a polynucleotide expression pattern which is detectable via the standard techniques of polymerase chain reaction (PCR), reverse transcriptase-(RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression level it is desired to detect.

A “target protein” refers to a polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e.g., gene or mRNA) whose expression level it is desired to detect.

“Complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

“Stringent conditions” refers to conditions under which a probe may hybridize to its target polynucleotide sequence, but to no other sequences. Stringent conditions are sequence-dependent (e.g., longer sequences hybridize specifically at higher temperatures). Generally, stringent conditions are selected to be about 5° C. lower than the thermal melting point (Tₘ) for the specific sequence at a defined ionic strength and pH. The Tₘ is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e.g., 10 to 50 nucleotides). Stringent conditions may also be achieved with the addition of destabilizing agents, such as formamide.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like. The invention also contemplates the use of radioactive isotopes, such as ³⁵S, ³²P, ³H, and the like as labels. Colorimetric labels such as colloidal gold or colored glass or plastic (e.g., polystyrene, polypropylene, latex) beads may also be utilized. See, e.g., U.S. Pat. Nos. 4,366,241; 4,277,437; 4,275,149; 3,996,345; 3,939,350; 3,850,752; and 3,817,837.

“Oligonucleotide probe” refers to an oligonucleotide that may recognize a particular target. Depending on context, the term “oligonucleotide probes” refers both to individual oligonucleotide molecules and to a collection of oligonucleotide molecules. In one aspect, an oligonucleotide probe comprises one or more polynucleotide sequences substantially identical to a target polynucleotide sequence or complementary sequence thereof, or portions of the target polynucleotide sequence or complementary sequence thereof.

“Protein-capture agent” refers to a molecule or a multi-molecular complex that can bind a protein to itself. In one embodiment, protein-capture agents bind their binding partners in a substantially specific manner. In one embodiment, protein-capture agents may exhibit a dissociation constant (KD) of less than about 10-6. The protein-capture agent may comprise a biomolecule such as a protein or a polynucleotide. The biomolecule may further comprise a naturally occurring, recombinant, or synthetic biomolecule. Examples of protein-capture agents include immunoglobulins, antigens, receptors, or other proteins, or portions or fragments thereof. Furthermore, protein-capture agents are understood not to be limited to agents that only interact with their binding partners through noncovalent interactions. Rather, protein-capture agents may also become covalently attached to the proteins with which they bind. For example, the protein-capture agent may be photocrosslinked to its binding partner following binding.

A “small molecule” comprises a compound or molecular complex, either synthetic, naturally derived, or partially synthetic, composed of carbon, hydrogen, oxygen, and nitrogen, which may also contain other elements, and which may have a molecular weight of less than about 15,000, less than about 14,000, less than about 13,000, less than about 12,000, less than about 11,000, less than about 10,000, less than about 9,000, less than about 8,000, less than about 7,000, less than about 6,000, less than about 5,000, less than about 4,000, less than about 3,000, less than about 2,000, less than about 1,000, less than about 900, less than about 800, less than about 700, less than about 600, less than about 500, less than about 400, less than about 300, less than about 200, or less than about 100.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

“BLAST” refers to Basic Local Alignment Search Tool, a technique for detecting ungapped sub-sequences that match a given query sequence. “BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence,” as understood herein, is a group of assembled overlapping sequences, particularly between sequences in one or more of the databases of the invention.

“SERPINE2” refers to serine (or cysteine) proteinase inhibitor, clade E, member 2. SERPINE2 is alternatively known to those skilled in the art as nexin; plasminogen activator inhibitor type 1, member 2; protease nexin 1 (PN1); protease inhibitor (PI7); and glia-derived nexin or glia-derived neurite promoting factor (GDN). See Crisp et al. J. BIOL. CHEM. 277(49): 47285-91 (2002); Strausberg et al. PROC. NATL. ACAD. SCI. USA 99(26): 16899-903 (2002); Carter et al. GENOMICS 27(1): 196-99 (1995); McGrogan et al. BIOTECH. 6: 172-177 (1998); Somer et al. BIOCHEM. 26(20): 6407-10 (1987); Gloor et al. CELL 47(5): 687-93 (1986). SERPINE2 is assigned to locus NM_{—}006216 in the GenBank database, ID 5270 in the LocusLink and EntrezGene Databases, ID Hs.21858 in the UniGene database, and ID 177010 in the On-line Mendelian Inheritance in Man (OMIM) database. The native SERPINE2 polynucleotides, and variants thereof of, are discussed in more detail below.

The terms “prognosis” and “prognose” refer to the act or art of foretelling the course of a disease. Additionally, the terms refer to the prospect of survival and recovery from a disease as anticipated from the usual course of that disease or indicated by special features of the individual case. Further, the terms refer to the art or act of identifying a disease from its signs and symptoms.

The terms “indicator” or “prognostic indicator” refer to anything that may serve as, or relate to, a ground or basis for a prognosis. These terms further refer to any grounds or basis of a differential diagnosis, including the results of testing and characterization of gene expression as described herein, and the distinguishing of a disease or condition from others presenting similar symptoms. Additionally, the terms “indicator” or “prognostic indicator” refer to any grounds or basis, including the results of testing and characterization of gene expression as described herein, which may be used to distinguish the probable course of a malignant disease.

### II. SERPINE2 Polynucleotides and Polypeptides and Variants Thereof

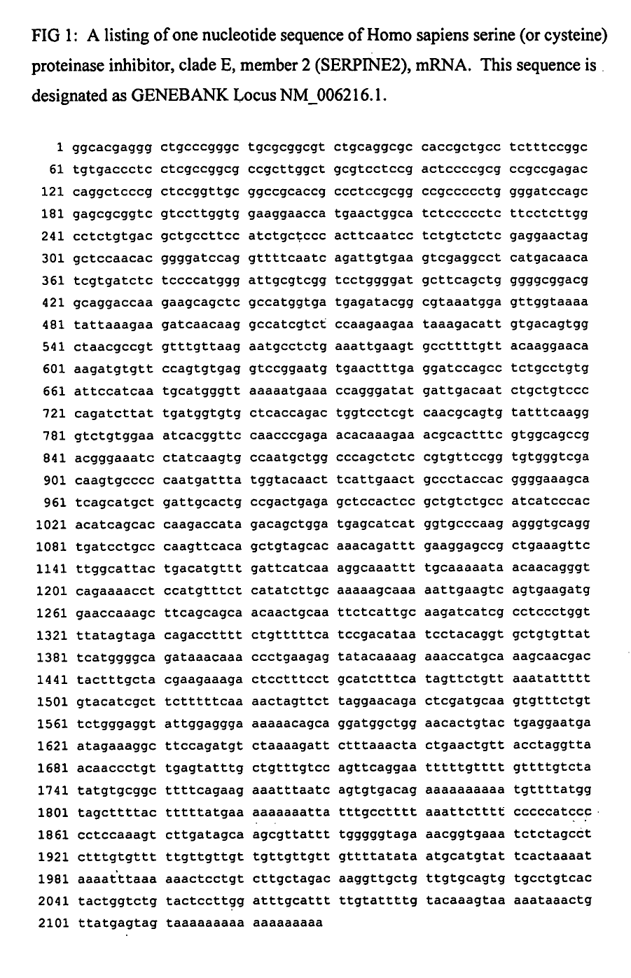

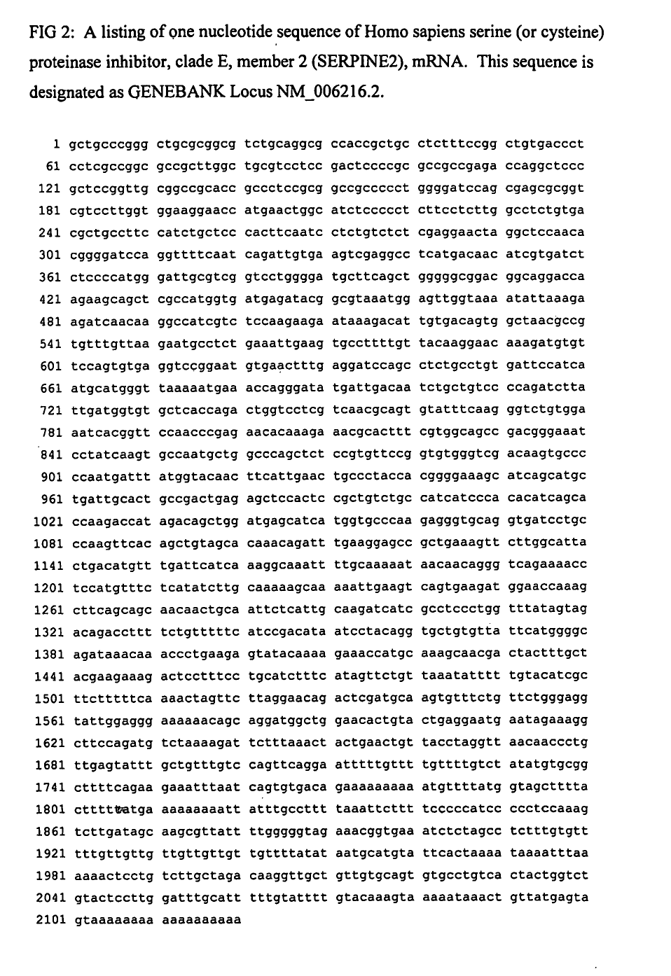

The term “SERPINE2 polynucleotide” refers generally to a polynucleotide that encodes a native SERPINE2 polypeptide or a fragment or variant thereof. In a specific embodiment, a SERPINE2 polynucleotide may encode native human SERPINE2 or a variant thereof. The polynucleotide sequence for human SERPINE2 is known in the art and is set forth in FIG. 1 and additionally set forth in FIG. 2. See Crisp et al. J. BIOL. CHEM. 277(49): 47285-91 (2002); Strausberg et al. PROC. NATL. ACAD. SCI. USA 99(26): 16899-903 (2002); Carter et al. GENOMICS 27(1): 196-99 (1995); McGrogan et al. BIOTECH. 6: 172-177 (1998); Somer et al. BIOCHEM. 26(20): 6407-10 (1987); Gloor et al. CELL 47(5): 687-93 (1986). SERPINE2 is assigned to locus NM_{—}006216 in the GenBank database, ID 5270 in the LocusLink and EntrezGene Databases, ID Hs.21858 in the UniGene database, and ID 177010 in the On-line Mendelian Inheritance in Man (OMIM) database. The SERPINE2 polynucleotides of the invention may be represented by these sequences or more particularly, by a polynucleotide sequence substantially identical to these polynucleotide sequences or complementary sequences thereof, or portions of these polynucleotide sequences or complementary sequences thereof.

Sequence identity or sequence similarity may be calculated based on a SERPINE2 reference sequence, which may be a subset of a larger SERPINE2 sequence, such as part of the coding region, flanking region, or a conserved motif. For example, as detailed above, the SERPINE2 reference sequence may be the coding region that encodes the extracellular domain of SERPINE2. Alternatively, the SERPINE2 reference sequence may be the coding region that encodes the intracellular domain of SERPINE2. A reference sequence may be at least about 18 contiguous nt long, alternatively at least about 30 nt long, and may extend to the complete sequence that is being compared. Moreover, the reference sequence may comprise the nucleotides that encode the amino acids which together constitute an epitope of SERPINE2. Algorithms for sequence analysis are known in the art, such as gapped BLAST, described in Altschul et al., 25 NUCL. ACIDS RES. 3389-3402 (1997).

Alternatively, sequence analysis may also be based on, but not limited to, the GCG Bestfit and Gap programs, which align two sequences either with the best local alignment (Bestfit) or a global alignment (gap), respectively. Local alignments are an optimal alignment of the best segment of similarity between two sequences. Optimal alignments are found by inserting gaps to maximize the number of matches using the local homology algorithm of Smith and Waterman.

SERPINE2 polynucleotides include naturally occurring variants, e.g., degenerate variants, allelic variants, and single nucleotide polymorphisms (SNPs), of the sequences provided herein. Additionally, the variants forms of SERPINE2 polypeptides contemplated by the invention further include, but are not limited to, mutants and fragments. Mutant SERPINE2 polynucleotide variants may result from nucleotide substitutions, deletions, and insertions. In general, variants of the SERPINE2 polynucleotides described herein have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

SERPINE2 variants, according to the invention, include those that are similar, identical, or substantially identical to the SERPINE2 polynucleotides provided herein. Polynucleotides having sequence similarity may be detected by hybridization under low stringency conditions, for example, at 50° C. and 10×SSC (0.9 M saline/0.09 M sodium citrate) and remain bound when subjected to washing at 55° C. in 1×SSC. Sequence identity and substantial sequence identity may be determined by hybridization under stringent conditions, for example, at 50° C. or higher and 0.1×SSC. Indeed, hybridization methods and conditions are well known in the art. See SAMBROOK ET AL., MOLECULAR CLONING: A LAB. MANUAL (2001); AUSBEL ET AL., CURRENT PROTOCOLS IN MOLECULAR BIOLOGY (1995).

The SERPINE2 polynucleotide compositions described herein may be used, for example, to produce polypeptides, which may be used to obtain anti-SERPINE2 immunoglobulins. In addition, the SERPINE2 polynucleotides may be used as probes, described in further detail below, to determine the presence or absence of SERPINE2 polynucleotides or variants thereof in a biological sample.

### III. SERPINE2 Polypeptides and Variants Thereof

In general, the term “SERPINE2 polypeptide,” as used herein, refers to both the full length polypeptide, as well as portions or fragments thereof.

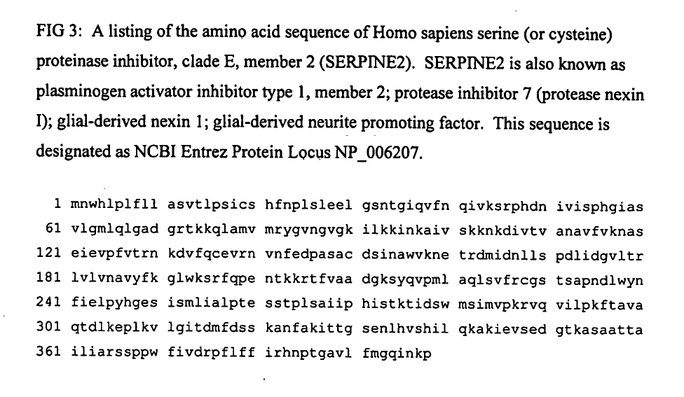

In one embodiment, SERPINE2 polypeptide comprises human SERPINE2. The amino acid sequence of human SERPINE2 is set forth in FIG. 3 and reported by McGrogan et al. BIOTECH. 6: 172-177 (1988); Sommer et al. BIOCHEM. 26(20): 6407-10 (1987); and Gloor et al. CELL 47(5): 687-93 (1986).

SERPINE2 polypeptides also encompass homologs of SERPINE2 polypeptides isolated from other species. Examples of SERPINE2 polypeptide homologs include those isolated from rodents, e.g., mice and rats, and domestic animals, e.g., horses, cows, dogs, and cats. There may be at least about 65% sequence identity, at least about 70% sequence identity, at least about 75% sequence identity, at least about 80% sequence identity, at least about 83% sequence identity, at least about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between SERPINE2 polypeptide homologs.

The invention also contemplates variants of SERPINE2 polypeptides, which include, but are not limited to, mutants, fragments, and fusions. In general, variants of the SERPINE2 polypeptides described herein have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

In one embodiment, the variant SERPINE2 polypeptide may be a mutant polypeptide. The mutations in the SERPINE2 polypeptide may result from, but are not limited to, amino acid substitutions, additions or deletions. The amino acid substitutions may be conservative amino acid substitutions or substitutions to eliminate non-essential amino acids. In general, conservative amino acid substitutions are those that preserve the general charge, hydrophobicity, hydrophilicity, and/or steric bulk of the amino acid substituted. In some mutant SERPINE2 polypeptides, amino acids may be substituted to alter a glycosylation site, a phosphorylation site or an acetylation site.

Importantly, variant polypeptides may be designed so as to retain or have enhanced biological activity of a particular region of the protein (e.g., a functional domain and/or, where the polypeptide is a member of a protein family, a region associated with a consensus sequence). Selection of amino acid alterations for production of variants may be based upon the accessibility (interior vs. exterior) of the amino acid (Go et al., 15 INT. J. PEPTIDE PROTEIN RES. 211 (1980)), the thermostability of the variant polypeptide (Querol et al., 9 PROT. ENG. 265 (1996)), desired glycosylation sites (Olsen and Thomsen, 137 J. GEN. MICROBIOL. 579 (1991)), desired disulfide bridges (Clarke et al., 32 BIOCHEMISTRY 4322 (1993); Wakarchuk et al., 7 PROTEIN ENG. 1379 (1994)), desired metal binding sites (Toma et al., 30 BIOCHEMISTRY 97 (1991); Haezerbrouck et al., 6 PROTEIN ENG. 643 (1993)), and desired substitutions within proline loops (Masul et al., 60 APPL. ENV. MICROBIOL. 3579 (1994)).

SERPINE2 polypeptide variants also include fragments of the polypeptides disclosed herein, particularly biologically active fragments and/or fragments corresponding to functional domains. SERPINE2 fragments may be at least about 10 amino acids to at least about 15 amino acids in length, at least about 50 amino acids in length, or at least about 300 amino acids in length or longer.

The SERPINE2 polypeptides of the invention are provided in a non-naturally occurring environment, e.g., are separated from their naturally occurring environment. In certain embodiments, the subject protein is present in a substantially purified form as defined above.

### IV. SERPINE2 Binding Partners

The invention relates to the use of a SERPINE2 binding partner to prognose a cancer characterized by overexpression and/or upregulation of SERPINE2. In this regard, the inventor has discovered that SERPINE2 is differentially expressed by several types of cancer cells, including colon, prostate, breast, thyroid, lung, ovarian, undifferentiated and leukemia cancer cells, and that upregulation of SERPINE2 negatively correlates with survival.

A. Immunoglobulins

SERPINE2 binding partners include immunoglobulins and functional equivalents of immunoglobulins that specifically bind to SERPINE2 polypeptides. The terms “immunoglobulin” and “antibody” are used interchangeably and in their broadest sense herein. Thus, they encompass intact monoclonal antibodies, polyclonal antibodies, multispecific antibodies (e.g., bispecific antibodies) formed from at least two intact antibodies, and antibody fragments, so long as they exhibit the desired biological activity. In one embodiment, the subject immunoglobulins comprise at least one human constant domain. In another embodiment, the SERPINE2 immunoglobulins comprise a constant domain that exhibits at least about 90-95% sequence identity with a human constant domain and yet retains human effector function. An immunoglobulin SERPINE2 binding partner or functional equivalent thereof may be human, chimeric, humanized, murine, CDR-grafted, phage-displayed, bacteria-displayed, yeast-displayed, transgenic-mouse produced, mutagenized, and randomized.

In a specific embodiment, the immunoglobulin SERPINE2 binding partner or functional equivalent thereof binds an epitope of SERPINE2 as expressed in a cancer cell. In another embodiment, the immunoglobulin SERPINE2 binding partner or functional equivalent thereof binds an peptide formed by the amino acid sequence of FIG. 3, or any portion thereof.

i. Antibodies Generally

The terms “antibody” and “immunoglobulin” cover fully assembled antibodies and antibody fragments that can bind antigen (e.g., Fab′, F′(ab)₂, Fv, single chain antibodies, diabodies), including recombinant antibodies and antibody fragments. Preferably, the immunoglobulins or antibodies are chimeric, human, or humanized.

The variable domains of the heavy and light chain recognize or bind to a particular epitope of a cognate antigen. The term “epitope” is used to refer to the specific binding sites or antigenic determinant on an antigen that the variable end of the immunoglobulin binds. Epitopes can be linear, i.e., be composed of a sequence of amino acid residues found in the primary SERPINE2 sequence. Epitopes also can be conformational, such that an immunoglobulin recognizes a 3-D structure found on a folded SERPINE2 molecule. Epitopes can also be a combination of linear and conformational elements. Further, carbohydrate portions of a molecule, as expressed by the target bearing tumor cells can also be epitopes.

Immunoglobulins are said to be “specifically binding” if: 1) they exhibit a threshold level of binding activity, and/or 2) they do not significantly cross-react with known related polypeptide molecules. The binding affinity of an immunoglobulin can be readily determined by one of ordinary skill in the art, for example, by Scatchard analysis (Scatchard, Ann. NY Acad. Sci. 51: 660-672, 1949). In some embodiments, the immunoglobulins of the present invention bind to SERPINE2 at least 10³, more preferably at least 10⁴, more preferably at least 10⁵, and even more preferably at least 10⁶ fold higher than to other proteins

ii. Polyclonal and Monoclonal Antibodies

Immunoglobulins of the invention may be polyclonal or monoclonal, and may be produced by any of the well known methods in this art.

Polyclonal antibodies are preferably raised in animals by multiple subcutaneous (sc), intraperitoneal (ip) or intramuscular (im) injections of the relevant antigen and an adjuvant. It may be useful to conjugate the relevant antigen to a protein that is immunogenic in the species to be immunized, In addition, aggregating agents such as alum are suitably used to enhance the immune response.

The term “monoclonal antibody” refers to an antibody obtained from a population of substantially homogeneous antibodies. Monoclonal antibodies are highly specific, being directed against a single antigenic site. Furthermore, in contrast to polyclonal antibody preparations that typically include different antibodies directed against different determinants, each monoclonal antibody is directed against a single determinant on the antigen.

In addition to their specificity, monoclonal antibodies are advantageous in that they may be synthesized while uncontaminated by other immunoglobulins. For example, monoclonal antibodies may be produced by the hybridoma method or by recombinant DNA methods. Monoclonal antibody SERPINE2 agents also may be isolated from phage antibody libraries.

iii. Chimeric and Humanized Antibodies

SERPINE2-binding immunoglobulins or antibodies can be “chimeric” in the sense that a variable region can come from a one species, such as a rodent, and the constant region can be from a second species, such as a human.

“Humanized” forms of non-human SERPINE2-binding antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody.

In general, the humanized antibody may comprise substantially all of at least one, and typically two, variable domains, in which all or substantially all of the hypervariable loops correspond to those of a non-human immunoglobulin and all or substantially all of the FRs are those of a human immunoglobulin sequence. In one embodiment, humanized antibodies comprise a humanized FR that exhibits at least 65% sequence identity with an acceptor (non-human) FR, e.g., murine FR. The humanized antibody also may comprise at least a portion of an immunoglobulin constant region (Fc), particularly a human immunoglobulin.

Methods for humanizing non-human antibodies have been described in the art. Preferably, a humanized antibody has one or more amino acid residues introduced into it from a source, which is non-human. These non-human amino acid residues are often referred to as “import” residues, which are typically taken from an “import” variable domain. Humanization may be essentially performed by substituting hypervariable region sequences for the corresponding sequences of a human antibody. Accordingly, such humanized antibodies are chimeric wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. The choice of human variable domains, both light and heavy, to be used in making the humanized antibodies is very important to reduce antigenicity.

Other methods generally involve conferring donor CDR binding affinity onto an antibody acceptor variable region framework. One method involves simultaneously grafting and optimizing the binding affinity of a variable region binding fragment. Another method relates to optimizing the binding affinity of an antibody variable region.

iv. Antibody Fragments

“Antibody fragments” comprise a portion of an intact antibody, preferably the antigen-binding or variable region thereof. Examples of antibody fragments include Fab, Fab′, F(ab′)², Fv fragments, diabodies, linear antibodies, single-chain antibody molecules, and multispecific antibodies formed from antibody fragments.

Papain digestion of antibodies produces two identical antigen-binding fragments, called “Fab” fragments, each with a single antigen-binding site, and a residual “Fc” fragment. The Fab fragments also contain the constant domain of the light chain and the first constant domain (CHI) of the heavy chain.

Pepsin treatment yields an F(ab′)² fragment that has two antigen-binding sites and is still capable of crosslinking antigen. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear at least one free thiol group. F(ab′)² antibody fragments originally were produced as pairs of Fab′ fragments which have hinge cysteines between them. Other chemical couplings of antibody fragments are well known in the art.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains that enables the scFv to form the desired structure for antigen binding. See PLUCKTHUN, 113 THE PHARMACOLOGY OF MONOCLONAL ANTIBODIES 269-315 (Rosenburg and Moore eds. 1994). See also WO 93/16185; U.S. Pat. Nos. 5,587,458 and 5,571,894.

Various techniques have been developed for the production of antibody fragments. Traditionally; these fragments were derived via proteolytic digestion of intact antibodies. However, these fragments may now be produced directly by recombinant host cells.

v. Conjugation and Labeling

Anti-SERPINE2 antibodies may be employed in their “naked” or unconjugated form, or may have other agents conjugated to them.

For examples the antibodies may be in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, and the like. Procedures for accomplishing such labeling are well known in the art.

vi. Bispecific Antibodies

Bispecific antibodies of the invention are small antibody fragments with two antigen-binding sites. Each fragment comprises a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen binding sites.

Methods for making bispecific antibodies are well known in the art. Traditional production of full length bispecific antibodies is based on the coexpression of two immunoglobulin heavy chain-light chain pairs, where the two chains have different specificities.

In another approach, antibody variable domains with the desired binding specificities (antibody-antigen combining sites) may be fused to immunoglobulin constant domain sequences. Specifically, the variable domains are fused with an immunoglobulin heavy chain constant domain, comprising at least part of the hinge, CH2, and CH3 regions. In one embodiment, the fusion protein comprises the first heavy-chain constant region (CHI) because it contains the site necessary for light chain binding. Polynucleotides encoding the immunoglobulin heavy chain fusions and, if desired, the immunoglobulin light chain, may be inserted into separate expression vectors and co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies also have been produced using leucine zippers. And single-chain Fv (sFv) dimers.

B. Non-Immunoglobulin Polypeptide Agents

In another embodiment, a SERPINE2 binding partner may be a peptide generated by rational design or by phage display. For example, the peptide may be a “CDR mimic” or immunoglobulin analogue based on the CDRs of an immunoglobulin.

C. Polynucleotide Binding Partners

Polynucleotide SERPINE2 binding partners may comprise one or more oligonucleotide probes. In the context of this invention, the term “oligonucleotide” refers to an oligomer or polymer of ribonucleic acid (RNA) or deoxyribonucleic acid (DNA) or variants thereof. Oligonucleotides may comprise naturally occurring nucleotides, sugars and covalent internucleoside (backbone) linkages as well as oligonucleotides having non-naturally occurring portions that function similarly. Such modified or substituted oligonucleotides possess desirable properties such as, for example, enhanced cellular uptake, enhanced affinity for polynucleotide target and increased stability in the presence of nucleases.

In general, oligonucleotide probes specifically hybridize with one or more polynucleotides encoding SERPINE2. With these target sites in mind, oligonucleotide probes that are sufficiently complementary to the target SERPINE2 polynucleotides must be chosen. There must be a sufficient degree of complementarity or precise pairing such that stable and specific binding occurs between the oligonucleotide and the SERPINE2 polynucleotide target. Importantly, the sequence of an oligonucleotide SERPINE2 probe need not be 100% complementary to that of its target SERPINE2 polynucleotide to be specifically hybridizable.

Probes specific to the SERPINE2 polynucleotides may be generated using the SERPINE2 polynucleotide sequences disclosed herein. Probes may be designed based on a subset of the SERPINE2 polynucleotide sequence, such as part of the coding region, flanking region, or a conserved motif. In one embodiment, the SERPINE2 probe may be designed from the coding region that encodes a protein domain or a portion of a protein domain as set forth in FIG. 3.

A SERPINE2 probe may comprise a contiguous sequence of nucleotides at least about 10 nt, at least about 12 nt, at least about 15 nt, at least about 16 nt, at least about 18 nt, at least about 20 nt, at least about 22 nt, at least about 24 nt, or at least about 25 nt in length that uniquely identifies a polynucleotide sequence. Moreover, a SERPINE2 probe may be at least about 30 nt, at least about 35 nt, at least about 40 nt, at least about 45, at least about 50 nt, at least about 55 nt, at least about 60 nt, at least about 70 nt, at least about 75 nt, at least about 80 nt, at least about 85 nt, at least about 90 nt, at least about 95 nt, at least about 100 nt, at least about 150 nt, at least about 200 nt, at least about 250 nt, at least about 300 nt, at least about 350 nt, at least about 400 nt, at least about 450 nt, at least about 500 nt, at least about 550 nt, at least about 600 nt, at least about 650 nt, at least about 700 nt, at least about 750 nt, at least about 800 nt, at least about 900 nt, at least about 950 nt, or at least about 1000 nt. Generally, a SERPINE2 probe may be at least about 10 nt to at least about 20 nt in length, at least about 50 nt to at least about 100 nt in length, at least about 10 to at least about 100 nt, or at least about 10 to at least about 1000 nt in length.

A SERPINE2 probe may exhibit less than about 99.99%, less than about 99.9%, less than about 99%, less than about 98%, less than about 97%, less than about 96%, less than about 95%, less than about 94, less than about 93%, less than about 92%, less than about 91%, less than about 90%, less than about 88%, less than about 85%, less than about 83%, less than about 80%, less than about 75%, less than about 70%, or less than about 65% sequence identity to any contiguous nucleotide sequence of more than about 15 nt. Furthermore, the probes may be synthesized chemically or may be generated from longer polynucleotides using restriction enzymes. In addition, the probes may be labeled with a radioactive, biotinylated, or fluorescent tag

Polynucleotides generally comprising at least 12 contiguous nt of a SERPINE2 polynucleotide are used for probes. A probe that hybridizes specifically to a SERPINE2 polynucleotide disclosed herein should provide a detection signal at least about 0.3-fold higher, at least about 0.5-fold higher, at least about 0.7-fold higher, at least about 0.8-fold higher, at least about 0.9-fold higher, at least about 1.0-fold higher, at least about 1.2-fold higher, at least about 1.4-fold higher, at least about 1.5-fold, at least about 1.6-fold higher, at least about 1.8-fold higher, at least about 2-fold higher, at least about 2.5-fold higher, at least about 3.0-fold higher, at least about 3.5-fold higher, at least about 4.0-fold higher, at least about 4.5-fold higher, at least about 5-fold higher, at least about 10-fold higher, or at least about 20-fold or more higher than the background hybridization provided with other unrelated sequences.

In addition to the sequences provided herein, SERPINE2 oligonucleotide probes, as well as oligonucleotide probes specific for other relevant genes, may be selected from a number of sources including polynucleotide databases such as GenBank, Unigen, HomoloGene, RefSeq, dbEST, and dbSNP. Wheeler et al., 29 NUCL. ACIDS RES. 11 16 (2001). More specifically, SERPINE2 oligonucleotide probes may be selected from FIG. 1, FIG. 2, locus NM_{—}006216 in the GenBank database, ID 5270 in the LocusLink Database, ID Hs.21858 in the UniGene database, and ID 177010 in the On-line Mendelian Inheritance in Man (OMIM) database. Generally, the probe is complementary to the reference sequence, preferably unique to the tissue or cell type (e.g., skeletal muscle, neuronal tissue) of interest, and preferably hybridizes with high affinity and specificity. Lockhart et al., 14 NATURE BIOTECHNOL. 1675-80 (1996). In addition, the oligonucleotide probe may represent non-overlapping sequences of the reference sequence, which improves probe redundancy resulting in a reduction in false positive rate and an increased accuracy in target quantitation. Lipshutz et al., 21 NATURE GENET. 20 24 (1999).

Generally, the oligonucleotide probes are generated by standard synthesis chemistries such as phosphoramidite chemistry (U.S. Pat. Nos. 4,980,460; 4,973,679; 4,725,677; 4,458,066; and 4,415,732; Beaucage and Iyer, 48 TETRAHEDRON 2223-2311 (1992)). Alternative chemistries that create non-natural backbone groups, such as phosphorothionate and phosphoroamidate may also be employed.

D. Small Molecules

Small molecules constitute another type of SERPINE2 binding agents. In general, small molecules comprise a compound or molecular complex, either synthetic, naturally derived, or partially synthetic, composed of carbon, hydrogen, oxygen, and nitrogen, which may also contain other elements, and which may have a molecular weight of less than about 15,000, less than about 14,000, less than about 13,000, less than about 12,000, less than about 11,000, less than about 10,000, less than about 9,000, less than about 8,000, less than about 7,000, less than about 6,000, less than about 5,000, less than about 4,000, less than about 3,000, less than about 2,000, less than about 1,000, less than about 900, less than about 800, less than about 700, less than about 600, less than about 500, less than about 400, less than about 300, less than about 200, or less than about 100.

E. Use and Formulation of SERPINE2 Binding Partners,

The many practical uses of SERPINE2 binding partners are apparent to those of skill in the art. For example, they may be used to purify, detect, and target SERPINE2 polypeptides, including both in vitro and in vivo diagnostic methods.

SERPINE2 binding partners generally are “substantially purified,” meaning separated and/or recovered from a component of their natural environment. Contaminant components of its natural environment are materials that would interfere with diagnostic uses for the SERPINE2 binding partner, and may include enzymes, hormones, and other proteinaceous or nonproteinaceous solutes. Ordinarily, an isolated agent will be prepared by at least one purification step. In one embodiment, the agent is purified to at least about 60%, at least about 65%, at least about 70%, at least about 75%, at least about 80%, at least about 85%, at least about 88%, at least about 90%, at least about 92%, at least about 95%, at least about 97%, at least about 98%, at least about 99%, at least about 99.9%, or at least about 99.99% by weight of SERPINE2 binding partner.

F. Modifications of SERPINE2 Binding Partners

Amino acid sequence variants of the polypeptide SERPINE2 binding partners of the invention may be prepared by introducing appropriate nucleotide changes into the polynucleotide that encodes the polypeptide SERPINE2 binding partner or by peptide synthesis. Such modifications include, for example, deletions from, and/or insertions into and/or substitutions of, residues within the amino acid sequences of the polypeptide SERPINE2 binding partner. Any combination of deletions, insertions, and substitutions may be made to arrive at the final construct, provided that the final construct decreases or inhibits the proliferation of a cancer characterized by overexpression and/or upregulation of SERPINE2.

Amino acid sequence insertions include amino-terminal and/or carboxyl-terminal fusions ranging in length from one residue to polypeptides containing a hundred or more residues, as well as intrasequence insertions of single or multiple amino acid residues. Examples of terminal insertions include a polypeptide SERPINE2 binding partner with an N-terminal methionyl residue or the polypeptide SERPINE2 binding partner fused to a cytotoxic polypeptide. Other insertional variants of the polypeptide SERPINE2 binding partner molecule include the fusion to the N- or C-terminus of the binding partner of an enzyme, or a polypeptide that increases the serum half-life of the binding partner.

Another type of polypeptide SERPINE2 binding partner variant is an amino acid substitution variant. These variants have at least one amino acid residue in the polypeptide SERPINE2 binding partner molecule replaced by a different residue. For example, the sites of greatest interest for substitutional mutagenesis of immunoglobulin SERPINE2 binding partners include the hypervariable regions, but FR alterations are also contemplated.

A useful method for the identification of certain residues or regions of the polypeptide SERPINE2 binding partner that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed binding partner variants screened for the desired activity.

Substantial modifications in the biological properties of the SERPINE2 polypeptide binding partner can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the polypeptide SERPINE2 binding partner also may be substituted, generally with serine, to improve the oxidative stability of the molecule and prevent aberrant crosslinking. Conversely, cysteine bond(s) may be added to the binding partner to improve its stability, particularly where the polypeptide SERPINE2 binding partner is an immunoglobulin fragment such as an Fv fragment.

Another type of substitutional variant involves substituting one or more hypervariable region residues of a parent immunoglobulin. Generally, the resulting variant(s), i.e., functional equivalents as defined above, selected for further development will have improved biological properties relative to the parent immunoglobulin from which they are generated. A convenient way for generating such substitutional variants is by affinity maturation using phage display. Briefly, several hypervariable region sites (e.g., 6-7 sites) are mutated to generate all possible amino substitutions at each site. The immunoglobulin variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the immunoglobulin-antibody complex to identify contact points between the immunoglobulin and antigen. Such contact residues and neighboring residues are candidates for substitution according to the techniques elaborated herein. Once generated, the panel of variants is subjected to screening as described herein and immunoglobulin with superior properties in one or more relevant assays may be selected for further development.

Another type of amino acid variant of the polypeptide SERPINE2 binding partner alters the original glycosylation pattern of the polypeptide SERPINE2 binding partner. An “altered glycosylation pattern” includes deleting one or more carbohydrate moieties found in the polypeptide SERPINE2 binding partner, and/or adding one or more glycosylation sites that are not present in the polypeptide SERPINE2 binding partner.

Glycosylation of polypeptides is typically either N-linked or O-linked. N-linked glycosylation refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the binding partner is conveniently accomplished by altering the amino acid sequence such that it contains one or more of the above-described tripeptide sequences.

O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The alteration may also be made by the addition of, or substitution by, one or more serine or threonine residues to the sequence of the original binding partner.

To increase the serum half life of an immunoglobulin SERPINE2 binding partner, one may incorporate a salvage receptor binding epitope into the SERPINE2 binding partner (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Polynucleotide molecules encoding amino acid sequence variants of the polypeptide SERPINE2 binding partners are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the polypeptide SERPINE2 binding partners.

### V. Diagnosis, Prognosis, and Assessment of Cancer Therapy

Experiments performed by the inventor demonstrate that the expression of SERPINE2 is a strong, negative prognostic indictor of cancer patient survival. In one embodiment, SERPINE2 expression can serve as a diagnostic or prognostic indicator of the course of a malignant disease. In a further embodiment, SERPINE2 expression can serve as a diagnostic or prognostic indicator of course of an individual afflicted with adult soft tissue sarcoma, colorectal carcinoma, and hepatocellular carcinoma.

A further aspect of the invention provides methods for using the SERPINE2 polynucleotides and polypeptides described herein to diagnose and prognose cancer. In specific non-limiting embodiments, the methods are useful for detecting SERPINE2-associated cancer cells, facilitating diagnosis of cancer and the severity of a cancer (e.g., tumor grade, tumor burden, disease stage, and the like) in a subject, facilitating a determination of the prognosis of a subject, determining the susceptibility to cancer in a subject, and assessing the responsiveness of the subject to therapy (e.g., by providing a measure of therapeutic effect through, for example, assessing tumor burden during or following a chemotherapeutic regimen). Such methods may involve detection of levels of SERPINE2 polynucleotides or SERPINE2 polypeptides in a patient biological sample, e.g., a suspected or prospective cancer tissue or cell. The detection methods of the invention may be conducted in vitro or in vivo, on isolated cells, or in whole tissues or a bodily fluid, e.g., blood, plasma, serum, urine, and the like. The detection method may or may not involve comparison of the levels of SERPINE2 polynucleotides or SERPINE2 polypeptides in a patient biological sample with that of levels of SERPINE2 polynucleotides or SERPINE2 polypeptides in a control biological sample. In one embodiment of the invention, this control biological sample may be an adjacent, noncancerous cell from the same tissue or cell type as the patient biological sample. In another embodiment, the control biological sample may be cells from a metastatic tumor from another tissue or cell type. Both of the foregoing embodiments may or may not involve biological samples and control biological samples from the same individual. Likewise in yet another embodiment of the invention, the control biological sample may consist of a population of cells in an organism or, alternatively, a collection of immortalized cells maintained in cell culture, or, alternatively, a collection of primary cells maintained ex vivo in cell culture.

In a particular embodiment, a method using SERPINE2 polynucleotides and polypeptides described herein to diagnose and prognose cancer entails (a) determining the level of expression of a SERPINE2 gene product within a patient tumor, relative to another gene product whose expression is invariant in different tissues, (b) determining the level of expression of a SERPINE2 gene product within nearby colon epithelium relative to the same invariant gene product used in (a), (c) comparing the levels of expression measured in (a) to (b), and (d) predicting overall survival or prognosis by comparing (c) to a standard curve relating SERPINE2 expression to survival, previously determined from measurements in a large number of patients. The relative level of SERPINE2 expression determined in (a) alone may also be used to predict survival.

In one embodiment of the invention, the aggressive nature and/or the metastatic potential of a cancer may be determined by comparing SERPINE2 polynucleotide and/or polypeptide levels to polynucleotide and/or polypeptide levels of another gene known to vary in cancerous tissue, e.g., expression of p53, DCC, ras, FAP. See, e.g., Fearon, 768 ANN. N.Y. ACAD. SCI. 101 (1995); Bodmer et al., 4(3) NAT. GENET. 217 (1994); Hamilton et al., 72 CANCER 957 (1993); and Fearon et al., 61(5) CELL 759 (1990). Thus, the expression of SERPINE2 polynucleotides and SERPINE2 polypeptides may be used to discriminate between normal and cancerous tissue, to discriminate between cancers with different cells of origin, and to discriminate between cancers with different potential metastatic rates, etc. For a review of cancer biomarkers, see Hanahan et al., 100 CELL 57-70 (2000).

In yet another embodiment, the levels of expression of the SERPINE2 polynucleotides, SERPINE2 polypeptides and SERPINE2 binding partners may be used as a prognostic indicator without comparison to a control sample. Overexpression and/or upregulation of SERPINE2 in patient biological samples alone can lead a person skilled in the art to a reasonable prognosis of the CRC patient condition. One example may be very low levels of SERPINE2 expression in a patient biological sample from a precancerous polyp. On the other hand, unexpectedly high levels of SERPINE2 obtained from a precancerous polyp can illicit an opposite prognosis.

In one embodiment, the SERPINE2 polynucleotides, SERPINE2 polypeptides and SERPINE2 binding partners may be used to detect, assess, and treat colon cancer. Colorectal cancer (CRC) is one of the most common neoplasms in humans and perhaps the most frequent form of hereditary neoplasia. Prevention and early detection are key factors in controlling and curing CRC. Precursors to this disease are often known as polyps, which are small, benign growths of cells that form on the inner lining of the colon. Over a period of time, some of these polyps accumulate additional mutations and become cancerous. Multiple familial CRC disorders have been identified and include Familial adenomatous polyposis (FAP); Gardner's syndrome; Hereditary nonpolyposis colon cancer (HNPCC); and Familial colorectal cancer in Ashkenazi Jews.

A. Detecting a SERPINE2 Polynucleotide in a Cell

Any of a variety of known methods may be used for detecting a SERPINE2 polynucleotide in a cell, including, detection of a SERPINE2 transcript by hybridization with a polynucleotide specific for a SERPINE2 transcript; detection of a SERPINE2 transcript by a polymerase chain reaction using specific oligonucleotide primers (RT-PCR); in situ hybridization of a cell using as a probe a polynucleotide that hybridizes to SERPINE2 that is differentially expressed in a colon cancer cell. The methods may be used to detect and/or measure SERPINE2 mRNA levels in a cancer cell. In some embodiments, the methods comprise a) contacting a sample with a SERPINE2 polynucleotide under conditions that allow hybridization; and b) detecting hybridization.

Detection of differential hybridization, when compared to a suitable control, is an indication of the presence in the sample of a SERPINE2 polynucleotide that is differentially expressed in a cancer cell. Appropriate controls include, for example, a sample which is known not to contain a SERPINE2 polynucleotide. Conditions that allow hybridization are known in the art. Detection may also be accomplished by any known method, including, but not limited to, in situ hybridization, PCR (polymerase chain reaction), RT-PCR (reverse transcription-PCR), and “Northern” or RNA blotting, or combinations of such techniques, using a suitably labeled polynucleotide. A variety of labels and labeling methods for polynucleotides are also known in the art and may be used in the assay methods of the invention. Specific hybridization may be determined by comparison to appropriate controls.

As will be readily appreciated by the ordinarily skilled artisan, the probe may be detectably labeled and contacted with, for example, a microarray comprising immobilized polynucleotides obtained from a test sample. Alternatively, the probe may be immobilized on a microarray and the test sample detectably labeled. These and other variations of the methods of the invention are well within the skill in the art and are within the scope of the invention.

Nucleotide probes may be used to detect expression of a gene corresponding to the provided SERPINE2 polynucleotide. In Northern blots, mRNA is separated electrophoretically and contacted with a probe. A probe is detected as hybridizing to an mRNA species of a particular size. The amount of hybridization may be quantitated to determine relative amounts of expression, for example under a particular condition. Probes are used for in situ hybridization to cells to detect expression. Probes may also be used in vivo for diagnostic detection of hybridizing sequences. Probes are typically labeled with a radioactive isotope. Other types of detectable labels may be used such as chromophores, fluorophores, and enzymes. Other examples of nucleotide hybridization assays are described in U.S. Pat. No. 5,124,246 and WO 92/02526.

PCR is another means for detecting small amounts of target SERPINE2 polynucleotides. See, e.g. Mullis et al., 155 METH. ENZYMOL. 335 (1987); U.S. Pat. Nos. 4,683,202; 4,683,195. Two primer polynucleotides that hybridize with the target SERPINE2 polynucleotides may be used to prime the reaction. The primers may comprise sequences within or 3′ and 5′ to the SERPINE2 polynucleotides provided in the sequence listing. Alternatively, if the primers are 3′ and 5′ to these polynucleotides, they need not hybridize to the polynucleotides or the complements. After amplification of the target with a thermostable polymerase, the amplified target polynucleotides may be detected by methods known in the art, e.g., Southern blot. SERPINE2 mRNA or cDNA may also be detected by traditional blotting techniques (e.g., Southern blot, Northern blot, etc.) described in SAMBROOK ET AL., MOLECULAR CLONING: A LAB. MANUAL (2001) (e.g., without PCR amplification). In general, mRNA or cDNA generated from mRNA using a polymerase enzyme may be purified and separated using gel electrophoresis, and transferred to a solid support, such as nitrocellulose. The solid support is exposed to a labeled probe, washed to remove any unhybridized probe, and duplexes containing the labeled probe are detected.

Methods using PCR amplification may be performed on the DNA from a single cell, although it is convenient to use at least about 10⁵ cells. The use of the polymerase chain reaction is described in Saiki et al., 239 SCIENCE 487 (1985), and a review of current techniques may be found in SAMBROOK ET AL., MOLECULAR CLONING: A LABORATORY MANUAL §§ 14.2-14.313 (2001). A detectable label may be included in the amplification reaction. Suitable detectable labels include fluorochromes, (e.g., fluorescein isothiocyanate (FITC), rhodamine, Texas Red, phycoerythrin, allophycocyanin, 6-carboxyfluorescein (6-FAM), 2′,7′-dimethoxy 4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g., ³²P, 35S′ 3H, etc.), and the like. The label may be a two stage system whereby the polynucleotides are conjugated to biotin, haptens, etc. having a high affinity binding partner, e.g., avidin, specific antibodies, etc., whereby the binding partner is conjugated to a detectable label. The label may be conjugated to one or both of the primers. Alternatively, the pool of nucleotides used in the amplification is labeled, so as to incorporate the label into the amplification product.

The invention provides methods for determining the presence, or absence, of a cancer characterized by overexpression and/or upregulation of SERPINE2. Specifically, in diagnosing a patient, the level of SERPINE2 expression in a biological sample obtained from a patient may be determined. Next, the level of SERPINE2 expression in the patient biological sample may be compared to the SERPINE2 expression level from a normal biological sample and correlated to a positive or negative diagnosis of cancer. A patient predisposition to a cancer characterized by overexpression and/or upregulation of SERPINE2 may be determined using a similar method. Moreover, the expression level of the biological samples may be determined using the polynucleotide and protein microarrays provided by the invention.

B. Detecting a SERPINE2 Polypeptide in a Cell

Any of a variety of known methods may be used for detecting a SERPINE2 polypeptide in a cell, including, but not limited to, immunoassay, using antibody specific for the encoded polypeptide, e.g., by enzyme-linked immunosorbent assay (ELISA), radioimmunoassay (RIA), and the like; and functional assays for the encoded polypeptide, e.g., biological activity.

For example, an immunofluorescence assay may be easily performed on cells without first isolating the encoded SERPINE2 polypeptide. The cells are first fixed onto a solid support, such as a microscope slide or microtiter well. This fixing step may permeabilize the cell membrane. The permeablization of the cell membrane permits the polypeptide-specific antibody to bind. Next, the fixed cells are exposed to an antibody specific for the encoded SERPINE2 polypeptide. To increase the sensitivity of the assay, the fixed cells may be further exposed to a second antibody, which is labeled and binds to the first antibody, which is specific for the encoded polypeptide. Typically, the secondary antibody is detectably labeled, e.g., with a fluorescent marker. The cells that express the encoded polypeptide will be fluorescently labeled and easily visualized under the microscope. See, e.g. Hashido et al., 187 BIOCHEM. BIOPHYS. RES. COMM. 1241-48 (1992).

As will be readily apparent to the ordinarily skilled artisan upon reading the present specification, the detection methods and other methods described herein may be readily varied. Such variations are within the intended scope of the invention. For example, in the above detection scheme, the probe for use in detection may be immobilized on a solid support, and the test sample contacted with the immobilized probe. Binding of the test sample to the probe may then be detected in a variety of ways, e.g., by detecting a detectable label bound to the test sample to facilitate detected of test sample-immobilized probe complexes.

The invention further provides methods for detecting the presence of and/or measuring a level of SERPINE2 polypeptide in a biological sample, using an antibody specific for SERPINE2. Specifically, the method for detecting the presence of SERPINE2 polypeptides in a biological sample may comprise the step of contacting the sample with a monoclonal antibody and detecting the binding of the antibody with the SERPINE2 in the sample. More specifically, the antibody may be labeled so as to produce a detectable signal using compounds including, but not limited to, a radiolabel, an enzyme, a chromophore and a fluorophore.

Detection of specific binding of an antibody specific for SERPINE2, or a functional equivalent thereof, when compared to a suitable control, is an indication that SERPINE2 polypeptides are present in the sample. Suitable controls include a sample known not to contain SERPINE2 polypeptides and a sample contacted with an antibody not specific for the encoded polypeptide, e.g., an anti-idiotype antibody. A variety of methods to detect specific antibody-antigen interactions are known in the art and may be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, 3-galactosidase, and the like); fluorescent labels (e.g., fluorescein isothiocyanate, rhodamine, phycoerythrin, and the like); fluorescence emitting metals (e.g., 112Eu, or others of the lanthanide series, attached to the antibody through metal chelating groups such as EDTA); chemiluminescent compounds (e.g., luminol, isoluminol, acridinium salts, and the like); bioluminescent compounds (e.g., luciferin, aequorin (green fluorescent protein), and the like). The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls and to appropriate standards.

In some embodiments, the methods are adapted for use in vivo, e.g., to locate or identify sites where SERPINE2-associated cancer cells are present. In these embodiments, a detectably-labeled moiety, e.g., an antibody, which is specific for SERPINE2 is administered to an individual (e.g., by injection), and labeled cells are located using standard imaging techniques, including, but not limited to, magnetic resonance imaging, computed tomography scanning, and the like. In this manner, SERPINE2 expressing cells are differentially labeled.

C. Kits

The detection methods may be provided as part of a kit. Thus, the invention further provides kits for detecting the presence and/or a level of a SERPINE2 polynucleotide expressed in a cancer cell (e.g., by detection of an mRNA encoded by the differentially expressed gene of interest), and/or a polypeptide encoded thereby, in a biological sample. Procedures using these kits may be performed by clinical laboratories, experimental laboratories, medical practitioners, or private individuals. The kits of the invention for detecting a polypeptide encoded by a SERPINE2 polynucleotide that is differentially expressed in a colon cancer cell may comprise a moiety that specifically binds the SERPINE2 polypeptide, which may be an antibody. The kits of the invention for detecting a SERPINE2 polynucleotide that is differentially expressed in a colon cancer cell may comprise a moiety that specifically hybridizes to such a polynucleotide. The kit may provide additional components that are useful in procedures, including, but not limited to, buffers, developing reagents, labels, reacting surfaces, means for detection, control samples, standards, instructions, and interpretive information.

D. Detecting SERPINE2 Using Arrays

The invention also relates to methods of using microarrays to analyze expression of the SERPINE2 gene.

Thus, the invention provides a method of using a microarray to prognose a cancer characterized by overexpression and/or upregulation of SERPINE2. The method includes (a) determining the level of expression of SERPINE2 in a biological sample obtained from a patient, using the microarray, (b) comparing the level of SERPINE2 expression in the patient biological sample to the level of SERPINE2 expression in a normal biological sample, and (c) correlating the level of SERPINE2 expression in the patient biological sample to a prognosis of the cancer.

Polynucleotide arrays provide a high throughput technique that can assay a large number of polynucleotides or polypeptides in a sample. This technology can be used as a tool to test for differential expression.

A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions.

Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734. In most embodiments, the “probe” is detectably labeled. In other embodiments, the probe is immobilized on the array and not detectably labeled.

Arrays can be used, for example, to examine differential expression of genes and can be used to determine gene function. For example, arrays can be used to detect differential expression of a gene corresponding to a polynucleotide described herein, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay, Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

A microarray contemplated by the invention also may contain any number of different proteins, amino acid sequences, polynucleotide sequences, or small molecules. In one embodiment, the microarrays may comprise all or a portion of the SERPINE2 protein, including functional derivatives, variants, analogs and portions thereof. The invention also contemplates microarrays comprising one or more antibodies or functional equivalents thereof that bind target proteins such as SERPINE2.

A protein-capture agent on the microarray may be any molecule or complex of molecules that has the ability to bind a target protein such as SERPINE2 and immobilize it to the site of the protein-capture agent on the microarray. In one aspect, the protein-capture agent binds its target protein in a substantially specific manner. For example, the protein-capture agent may be a protein whose natural function in a cell is to specifically bind another protein, such as an antibody or a receptor. Alternatively, the protein-capture agent may be a partially or wholly synthetic or recombinant protein that specifically binds a target protein.

Moreover, the protein-capture agent may be a protein that has been selected in vitro from a mutagenized, randomized, or completely random and synthetic library by its binding affinity to a specific target protein or peptide target. The selection method used may be a display method such as ribosome display or phage display. Alternatively, the protein-capture agent obtained via in vitro selection may be a DNA or RNA aptamer that specifically binds a protein target. See, e.g., Potyrailo et al., 70 ANAL. C HEM. 3419-25 (1998); Cohen, et al., 94 PROC. N ATL. ACAD. SCI. USA 14272-7 (1998); Fukuda, et al., 37 NUCL. ACIDS SYMP. SER., 237-8 (1997). Alternatively, the in vitro selected protein-capture agent may be a polypeptide. Roberts and Szostak, 94 PROC. NATL. ACAD. SCI. USA 12297-302 (1997). In yet another embodiment, the protein-capture agent may be a small molecule that has been selected from a combinatorial chemistry library or is isolated from an organism.

In a particular embodiment, however, the protein-capture agents are proteins. The protein-capture agents may be antibodies or antibody fragments. Although antibody moieties are exemplified herein, it is understood that the present microarrays and methods may be advantageously employed with other protein-capture agents.

The antibodies or antibody fragments of the microarray may be single-chain Fvs, Fab fragments, Fab′ fragments, F(ab′)2 fragments, Fv fragments, dsFvs diabodies, Fd fragments, full-length, antigen-specific polyclonal antibodies, or full-length monoclonal antibodies. In a specific embodiment, the protein-capture agents of the microarray are monoclonal antibodies, Fab fragments or single-chain Fvs.

The antibodies or antibody fragments may be monoclonal antibodies, even commercially available antibodies, against known, well-characterized proteins. Alternatively, the antibody fragments may be derived by selection from a library using the phage display method. If the antibody fragments are derived individually by selection based on binding affinity to known proteins, then the target proteins of the antibody fragments are known. In an alternative embodiment of the invention, the antibody fragments are derived by a phage display method comprising selection based on binding affinity to the (typically, immobilized) proteins of a cellular extract or a biological sample. In this embodiment, some or many of the antibody fragments of the microarray would bind proteins of unknown identity and/or function.

E. Biological Samples

Biological samples for use in the methods described herein may be isolated from several sources including, but not limited to, a patient or a cell line. Patient samples may include blood, urine, amniotic fluid, plasma, semen, bone marrow, and tissues. Once isolated, total RNA or protein may be extracted using methods well known in the art. For example, target samples may be generated from total RNA by dT-primed reverse transcription producing cDNA. See, e.g., SAMBROOK ET AL., MOLECULAR CLONING: A LAB. MANUAL (2001); and AUSUBEL ET AL., CURRENT PROTOCOLS IN MOLECULAR BIOLOGY, John Wiley & Sons, Inc. (1995). The cDNA may then be transcribed to cRNA by in vitro transcription resulting in a linear amplification of the RNA. The target samples may be labeled with, for example, a fluorescent dye (e.g., Cy3-dUTP) or biotin. The labeled targets may be hybridized to the microarray. Laser excitation of the target samples produces fluorescence emissions, which are captured by a detector. This information may then be used to generate a quantitative two-dimensional fluorescence image of the hybridized targets.

Gene expression profiles of a particular tissue or cell type may be generated from RNA (i.e., total RNA or mRNA). Reverse transcription with an oligo-dT primer may be used to isolate and generate mRNA from cellular RNA. To maximize the amount of sample or signal, labeled total RNA may also be used. The RNA may be fluorescently labeled or labeled with a radioactive isotope. For radioactive detection, a low energy emitter, such as ³³P-dCTP, is preferred due to close proximity of the oligonucleotide probes on the support. The fluorophores, Cy3-dUTP or Cy5-dUTP, may used for fluorescent labeling. These fluorophores demonstrate efficient incorporation with reverse transcriptase and better yields. Furthermore, these fluorophores possess distinguishable excitation and emission spectra. Thus, two samples, each labeled with a different fluorophore, may be simultaneously hybridized to a microarray.

Typically, the polynucleotide sample may be amplified prior to hybridization. Amplification methods include, but are not limited to PCR (INNIS ET AL., PCR PROTOCOLS: A GUIDE TO METHODS & APPLICATION (1990)), ligase chain reaction (LCR) (Wu & Wallace, 4(4) GENOMICS 560-69 (1989); Landegren et al., 241(4869) SCIENCE 1077-80 (1988); Barringer et al., 89(1) GENE 117-22 (1990)), transcription amplification (Kwoh et al., 86(4) PNAS 1173-77 (1989)), and self-sustained sequence replication (Guatelli et al., 87(5) PNAS 1874-78 (1990)).

The target polynucleotides may be labeled at one or more nucleotides during or after amplification. Labels suitable for use include labels detectable by spectroscopic, photochemical, biochemical, immunochemical, electrical, optical, or chemical means. In one embodiment, the detectable label is a luminescent label, such as fluorescent labels, chemiluminescent labels, bioluminescent labels, and colorimetric labels. In a specific embodiment, the label is a fluorescent label such as fluorescein, rhodamine, lissamine, phycoerythrin, polymethine dye derivative, phosphor, or Cy2, Cy3, Cy3.5, Cy5, Cy5.5, Cy7. Commercially available fluorescent labels include fluorescein phosphoramidites such as Fluoreprime (Pharmacia, Piscataway, N.J.), Fluoredite (Millipore, Bedford, Mass.), and FAM (ABI, Foster City, Calif.). Other labels include biotin for staining with labeled streptavidin conjugate, magnetic beads (e.g., Dynabeads), fluorescent dyes (e.g., texas red, rhodamine, green fluorescent protein), radiolabels (e.g., 3H, 125I, 35S, 14C, or 32P), enzymes (e.g., horseradish peroxidase, alkaline phosphatase), and colorimetric labels such as colloidal gold or colored glass or plastic (e.g., polystyrene, polypropylene, latex) beads (see, e.g., U.S. Pat. Nos. 4,366,241; 4,277,437; 4,275,149; 3,996,345; 3,939,350; 3,850,752; and 3,817,837).

### VI. Identifying SERPINE2 Binding Partners

The invention further provides methods for identifying SERPINE2 binding partners. Such methods involve, for example, the screening of libraries containing candidate SERPINE2 binding partners and analyzing the results of direct binding, competitive binding, and other assays.

A. High-Throughput Screening of Combinatorial Libraries

High-throughput screening methods involve a library containing a large number of “candidate compounds.” Such “combinatorial chemical libraries” are then screened in one or more assays to identify those library members, i.e., particular chemical species or subclasses, which display a desired characteristic activity. The identified compounds may serve as conventional lead compounds or may themselves be used as potential or actual therapeutics. Because of the ability to test large numbers quickly and efficiently, high-throughput screening methods are replacing conventional lead compound identification methods.

Generally, a combinatorial chemical library is a collection of diverse chemical compounds generated by combining a number of chemical “building blocks” via chemical synthesis or biological synthesis. Millions of chemical compounds may be synthesized through combinatorial mixing of chemical building blocks. In fact, the systematic, combinatorial mixing of 100 interchangeable chemical building blocks results in the theoretical synthesis of 100 million tetrameric compounds or 10 billion pentameric compounds. In a specific example, a linear combinatorial chemical library such as a polypeptide library may be formed by combining a set of amino acids in every possible way for a given compound length, i.e., the number of amino acids in a polypeptide compound.

Any type of molecule that is capable of binding to SERPINE2 may be present in the compound library. For example, combinatorial compound libraries may contain naturally-occurring molecules such as carbohydrates, monosaccharide, oligosaccharides, polysaccharides, amino acids, peptides, oligopeptides, polypeptides, proteins, nucleosides, nucleotides, oligonucleotides, polynucleotides including DNA, RNA, and fragments thereof, lipids, retinoids, steroids, glycopeptides, glycoproteins, glycolipids, proteoglycans; analogs or derivatives of naturally-occurring molecules such as peptidomimetics; non-naturally occurring molecules such as “small molecule” organic compounds; organometallic compounds; inorganic ions; and mixtures thereof.

In one embodiment, a combinatorial library may be a peptide library. See, e.g., U.S. Pat. No. 5,010,175; Furka, 37 INT. J PEPT. PROT. RES. 487-93 (1991); Houghton et al., 354 NATURE 84-88 (1991). Other combinatorial libraries that may be probed to identify SERPINE2 binding partners include polynucleotide libraries; peptide nucleic acid libraries (U.S. Pat. No. 5,539,083); antibody libraries (Vaughn et al., 14(3) NATURE BIOTECH. 309-14 (1996); WO 96/10287); carbohydrate libraries (Liang et al., 274 SCIENCE 1520-22 (1996); U.S. Pat. No. 5,593,853); and small organic molecule libraries (Chen et al., 116 J. AMER. CHEM. SOC. 2661 (1994).

Other compound libraries may be generated. Such compounds may include, but are not limited to, peptoids (WO 91/19735); encoded peptides (WO 93/20242); random biooligomers (WO 92/00091); diversomers such as hydantoins, benzodiazepines and dipeptides (Hobbs et al., 90 PROC. NAT. ACAD. SCI. USA 6909-13 (1993); U.S. Pat. No. 5,288,514); vinylogous polypeptides (Llagihara et al., 114 J. AMER. CHEM. SOC. 6568 (1992)); nonpeptidal peptidomimetics with a Beta D Glucose scaffolding (Hirschmann et al., 114 J. AMER. CHEM. SOC. 9217-18 (1992)); oligocarbamates (Cho et al., 261 SCIENCE 1303 (1993)); peptidyl-phosphonates (Campbell et al., 59 J. ORG. CHEM. 658 (1994)); isoprenoids (U.S. Pat. No. 5,569,588); thiazolidinones and metathiazanones (U.S. Pat. No. 5,549,947), pyrrolidines (U.S. Pat. Nos. 5,525,735; 5,519,134), and morpholino compounds (U.S. Pat. No. 5,506,337).

The compound libraries employed in the invention may be prepared or obtained by any means including, but not limited to, combinatorial chemistry techniques, fermentation methods, plant and cellular extraction procedures and the like. Methods for making combinatorial libraries are well known in the art. See, e.g., Carell et al., 3 CHEM. BIOL. 171-83 (1995); Felder, 48 CHIMIA 512-41 (1994); Gallop et al., 37 J. MED. CHEM. 1233-51 (1994); Gordon et al., 37 J. MED. CHEM. 1385-1401 (1994); Houghten, 9 TRENDS GENET. 235-39 (1993); Brenner et al., 89 PROC. NATL. ACAD. SCI. USA 5381-83 (1992); Houghten et al., 354 NATURE 84-86 (1991); Lam et al., 354 NATURE 82-84 (1991); Cwirla et al., 87 BIOCHEMISTRY 6378-82 (1990); and Lebl et al., 37 BIOPOLYMERS 177-98 (1995). Devices for the preparation of combinatorial libraries are commercially available. See, e.g., 390 MPS (Advanced Chem. Tech., Louisville Ky.); Symphony (Rainin, Wobum, Mass.); 433A (Applied BioSystems, Foster City, Calif.); and 9050 Plus (Millipore, Bedford, Mass.).

Alternatively, numerous combinatorial libraries are themselves commercially available. See, e.g., ComGenex Corp., Princeton, N.J.; Asinex Ltd., Moscow, Russia; Tripos, Inc., St. Louis, Mo.; ChemStar, Ltd, Moscow, Russia; 3D Pharmaceuticals, Inc., Exton, Pa.; and Martek Biosciences Corp., Columbia, Md.

High-throughput screening systems are commercially available. See, e.g., Zymark Corp., Hopkinton, Mass.; Beckman Instruments, Inc. Fullerton, Calif.; and Precision Systems, Inc., Natick, Mass. These systems typically automate entire procedures including all simple and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for the various high-throughput systems and methods.

The compound to be tested may include, not only known ligands, such as angiotensins, bombesins, canavinoids, cholecystokinins, glutamine, serotonin, melatonins, neuropeptides Y, opioids, purine, vasopressins, oxytocins, VIP (vasoactive intestinal and related peptides), somatostatins, dopamine, motilins, amylins, bradykinins, CGRP (calcitonin gene related peptides), adrenomedullins, leukotrienes, pancreastatins, prostaglandins, thromboxanes, adenosine, adrenaline, interleukins, α- and β-chemokines (IL-8, GROα, GROβ, GRO, NAP-2, ENA-78, PF4, IP10, GCP-2, MCP-1, HC14, MCP-3, I-309, MIP1α, MIP-1β, RANTES, and the like), endothelins, enterogastrins, histamine, neurotensins, TRH, pancreatic polypeptides, galanin, modified derivatives thereof, analogues thereof, family members thereof and the like, but also tissue extracts, cell culture supernatants, and so forth, of any organism including, but not limited to, plants and mammals (such as mice, rats, swine, cattle, sheep, monkeys and humans). For example, the biological sample extract, or cell culture supernatant, is added to SERPINE2 for measurement of the cell stimulating activity, and so forth, and fractionated by relying on the measurements whereupon a single ligand can be finally obtained.

B. Additional Methods for Identifying SERPINE2 Binding Partners

Another way of identifying SERPINE2 binding partners can be in vitro screening to identify compounds that selectively bind the protein. The various combinatorial libraries discussed in detail above may be used to screen candidate SERPINE2 binding partners.

A composition comprising SERPINE2 may be used in a binding assay to detect and/or identify binding partners that can bind to the protein. Compositions suitable for use in a binding assay include, for example, an isolated SERPINE2 protein or a functional equivalent thereof, cells that naturally express a mammalian SERPINE2 and recombinant cells comprising an exogenous polynucleotide sequence that encodes a mammalian SERPINE2.

Candidate SERPINE2 binding partners may identified by direct binding assays. Sees e.g. Schoemaker et al., 285 J. PHARMACOL. EXP. THER. 61-69 (1983). In one embodiment, a labeled candidate binding partner is contacted with SERPINE2 and the amount of labeled candidate binding partner binding with SERPINE2 is measured. In another embodiment, a labeled candidate binding partner is contacted with a cell that naturally expresses SERPINE2 on its surface and the amount of labeled candidate binding partner binding with the cells is measured. Alternatively, a cell transfected with an expression vector comprising a gene encoding SERPINE2 may be used. In yet another embodiment, a labeled candidate binding partner is contacted with a fraction isolated from cells that naturally expresses SERPINE2 and the amount of labeled candidate binding partner binding with the SERPINE2 contained within the fraction is measured. Alternatively, the fractions may be prepared from cells transfected with an expression vector comprising a gene encoding SERPINE2. In both embodiments, the fractions comprise SERPINE2 or a functional equivalent thereof.

In practicing the methods described herein, the SERPINE2 proteins, cells expressing SERPINE2, and fractions comprising SERPINE2 may be suspended in a buffer suitable for identifying binding partners of SERPINE2. Any buffer that does not inhibit the binding of the binding partner to SERPINE2 may be used and may include buffers such as Tris-HCl buffer or phosphate buffer of pH 4-10 (often, around pH 6-8). In conducting the screening, a suitable buffer is used that does not show toxicity to cells during the incubation period with the candidate binding partner. Alternatively, binding assay may be performed in a suitable growth medium.

In addition, a surface-active agent such as CHAPS, Tween® 80, digitonin, deoxycholate, and/or various other proteins such as bovine serum albumin (BSA), gelatin, and the like, may be added to the buffer to decrease nonspecific binding. Furthermore, a protease inhibitor such as PMSF, leupeptin, or pepstatin may be added to prevent protease digestion of the SERPINE2 and the candidate peptide or polypeptide binding partners. A labeled candidate binding partner, for example a radioactive label from about 5,000 cpm to about 500,000 cpm, may be added to about 0.001 ml to about 10 ml of the SERPINE2 solution.

The binding assay may be performed at about 0° C. to about 50° C., preferably at about 4° C. to about 37° C., for about twenty minutes to about twenty-four hours, preferably about thirty minutes to about three hours. After the reaction, the solution may be filtered through a glass fiber filter, a filter paper or the like; washed with a suitable amount of the buffer; and the radioactivity retained in the filter is measured by means of a liquid scintillation counter or a gamma-counter. Excess label may be used in a separate reaction to determine the amount of nonspecific binding.

Thus, in one embodiment, the invention provides a method of screening for a SERPINE2 binding partner comprising the steps of culturing a cell line transfected with an expression vector comprising a gene encoding SERPINE2 to express SERPINE2 in media containing at least one candidate binding partner of SERPINE2 and measuring the binding of at least one candidate binding partner to the SERPINE2 proteins produced by the cell line. In a specific embodiment, the cell line is derived from a mammal, preferably a human. Moreover, SERPINE2 may be encoded by a polynucleotide sequence substantially identical to a polynucleotide sequence or complementary sequence thereof, or portions of the polynucleotide sequence or complementary sequence thereof, selected from the group consisting of FIG. 1 or FIG. 2. In another embodiment, the candidate binding partner is labeled. The label may be, but is not limited to a radiolabel, an enzyme, a chromophore or a fluorophore.

In yet another embodiment, the invention provides a method of screening for a SERPINE2 binding partner comprising the steps of contacting at least one candidate binding partner with a protein domain of SERPINE2 under conditions whereby the at least one candidate binding partner can bind the protein domain of SERPINE2; and detecting the binding of the at least one candidate binding partner to the extracellular domain of SERPINE2. In a specific embodiment, SERPINE2 may be encoded by a polynucleotide sequence substantially identical to a polynucleotide sequence or complementary sequence thereof, or portions of the polynucleotide sequence or complementary sequence thereof, selected from the group consisting of FIG. 1 or FIG. 2. In another embodiment, the candidate binding partner is labeled. The label may be, but is not limited to a radiolabel, an enzyme, a chromophore or a fluorophore. In a specific embodiment, SERPINE2 is located within a cell expressing SERPINE2. Alternatively, the protein domain of SERPINE2 may be located in a fraction isolated from a cell expressing SERPINE2.

In an alternative embodiment, candidate binding partners may be identified by indirect, e.g., competitive, binding. These types of assays may be used to assess the binding affinity of candidate binding partners. In one embodiment, the method of detecting or identifying a SERPINE2 binding partner comprises a competitive binding assay in which the ability of a candidate binding partner to inhibit the binding of a known binding partner or known ligand, e.g., an antibody, is assessed. For example, the known binding partner or known ligand may be labeled with a suitable label as described herein, and the amount of labeled known binding partner or known ligand required to saturate the SERPINE2 present in the assay may be determined. A saturating amount of labeled known binding partner or known ligand and various amounts of a candidate binding partner may be contacted with a composition comprising a mammalian SERPINE2 under conditions suitable for binding and complex formation determined. In this type of assay, a decrease in the amount of complex formed between the labeled known binding partner or known ligand and SERPINE2 indicates that the candidate binding partner binds to SERPINE2.

The formation of a complex between the known binding partner, for example, a known ligand, and SERPINE2 may be detected or measured directly or indirectly using any suitable method. For example, the known binding partner may be labeled with a suitable label and the formation of a complex can be determined by detection of the label. The specificity of the complex may be determined using a suitable control such as excess unlabeled known binding partner or known ligand or label alone. Labels suitable for use in detection of a complex between an agent and a mammalian SERPINE2 include, for example, a radioisotope, an epitope label (tag), an affinity label (e.g., biotin, avidin), a spin label, an enzyme, a fluorescent group or a chemiluminescent group. If a label is not employed, complex formation may be determined by surface plasma resonance or other suitable methods.

The capacity of the candidate binding partner to inhibit the formation of a complex between the known binding partner and a mammalian SERPINE2 may be reported as the concentration of candidate binding partner required for 50% inhibition (IC50 values) of specific binding of labeled known binding partner or known ligand. In one embodiment, specific binding is defined as the total binding (e.g., total label in complex) minus the non-specific binding. Non-specific binding may be defined as the amount of label still detected in complexes formed in the presence of excess unlabeled known binding partner or known ligand. Known binding partner, for example, known ligands, which are suitable for use in the methods described herein include molecules and compounds that specifically bind to a mammalian SERPINE2 such as an immunoglobulin.

Thus, in one embodiment, the invention provides a method of determining the ability of a drug to inhibit ligand binding to SERPINE2 comprising the steps of culturing a cell line transfected with an expression vector comprising a gene encoding SERPINE2 to express it in the presence of ligand and in the presence of both ligand and drug and comparing the level of binding of the ligand to expressed SERPINE2 to the level of binding of the ligand to expressed SERPINE2 in the presence of the drug, wherein a lower level of ligand binding in the presence of the drug indicates that the drug is an inhibitor of ligand binding and thus, may have applicability in preventing, ameliorating, treating or delaying the onset of a cancer. In a specific embodiment, SERPINE2 may be encoded by a polynucleotide sequence substantially identical to a polynucleotide sequence or complementary sequence thereof, or portions of the polynucleotide sequence or complementary sequence thereof, selected from the group consisting of FIG. 1 or FIG. 2. In this particular method, the cell line may be from a mammal, preferably a human. Moreover, the ligand and/or the drug may be labeled.

Similarly, the invention provides another method of determining the ability of a drug to inhibit ligand binding to SERPINE2 comprising the steps of incubating fractions isolated from a cultured cell line transfected with an expression vector comprising a gene encoding SERPINE2, wherein the membranes contain the expressed SERPINE2, in the presence of ligand and in the presence of both the ligand and the drug; and comparing the level of binding of the ligand to the expressed SERPINE2 to the level of binding of the ligand to the expressed SERPINE2 in the presence of the drug, wherein a lower level of ligand binding in the presence of the drug indicates that the drug is an inhibitor of ligand binding. In a specific embodiment, SERPINE2 may be encoded by a polynucleotide sequence substantially identical to a polynucleotide sequence or complementary sequence thereof, or portions of the polynucleotide sequence or complementary sequence thereof, selected from the group consisting of FIG. 1 or FIG. 2. In another embodiment, the candidate binding partner is labeled. The label may be, but is not limited to a radiolabel, an enzyme, a chromophore or a fluorophore.

Alternatively, binding partners or targets that bind to SERPINE2 may be identified using a yeast two-hybrid system (Fields et al., 340 NATURE 245-246 (1989)). In this system, an expression unit encoding a fusion protein comprising one subunit of a two subunit transcription factor and SERPINE2 may be introduced and expressed in a yeast cell. The cell may be modified further to contain (1) an expression unit encoding a detectable marker whose expression requires the two subunit transcription factor for expression and (2) an expression unit that encodes a fusion protein comprising the second subunit of the transcription factor and a cloned segment of DNA. If the cloned segment of DNA encodes a protein that binds to SERPINE2, the expression results in the interaction of SERPINE2 and the encoded protein. This interaction also brings the two subunits of the transcription factor into binding proximity allowing reconstitution of the transcription factor, which results in the expression of the detectable marker. The yeast two hybrid system is particularly useful in screening a library of cDNA encoding segments for cellular binding partners of SERPINE2.

### VII. Vectors, Host Cells and Protein Production

The present invention also provides vectors containing SERPINE2 polynucleotides, host cells containing such vectors, and the production of SERPINE2 polypeptides by recombinant techniques. The vector may be, for example, a phage, plasmid, viral, or retroviral vector. Retroviral vectors may be replication competent or replication defective. In the latter case, viral propagation generally will occur only in complementing host cells.

SERPINE2 polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria.

Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. 5 Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNHSA, pNH 16a, pNH 18A, pNH46A, available from Stratagene Cloning Systems, Inc.; and ptrc99a, pKK223-3, pKK233-3, pDR540, pRITS available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carload, Calif.). Other suitable vectors will be readily apparent to the skilled artisan.

Nucleic acids of interest may be cloned into a suitable vector by route methods. Suitable vectors include plasmids, cosmids, recombinant viral vectors e.g. retroviral vectors, YACs, BACs and the like, phage vectors.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A SERPINE2 polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

SERPINE2 polypeptides can also be recovered from products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast higher plant, insect, and mammalian cells. Depending upon the host employed in a recombinant production procedure, the SERPINE2 polypeptides may be glycosylated or may be non-glycosylated. In addition, SERPINE2 polypeptides may also include an initial modified methionine residue, in some cases as a result of host-5 mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express SERPINE2 polypeptides in a eukaryotic system. Pichia pastoris is a methylotrophic yeast which can metabolize methanol as its sole carbon source. A main step in the methanol metabolization pathway is the oxidation of methanol to formaldehyde using O2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al, Mol. Cell. Biol. 5:1111-21 (1985); Koutz, P. J, et al, Yeast 5:167-77 (1989); Tschopp, J. F., et al, Nucl. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a SERPINE2 polypeptide, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a SERPINE2 polypeptide by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pP!C9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, a SERPINE2 polynucleotide, may be achieved by cloning the heterologous SERPINE2 polynucleotide into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with SERPINE2 polynucleotides, and which activates, alters, and/or amplifies endogenous polynucleotides. For example, techniques known in the art may be used to operably associate heterologous control regions (e.g., promoter and/or enhancer) and endogenous polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather. Biol. Reprod. 23:243-251 (1980)), —monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (Hep G2, HB 8065); mouse mammary tumor (MMF 060562, ATCC CCL51); TRI cells (Mather et al., Annals N.Y. Acad. Sci. 383:44-68 (1982)); MRC 5 cells; FS4 cells; and a human hepatoma line (Hep G2).

In addition, SERPINE2 polypeptides can be chemically synthesized using techniques known in the art (see e.g., Creighton, 1983, Proteins: Structures and Molecular Principles, W.H. Freeman & Co., N.Y., and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, α-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, 5 hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

Non-naturally occurring variants may be produced using art-known mutagenesis techniques, which include, but are not limited to oligonucleotide mediated mutagenesis, alanine scanning, PCR mutagenesis, site directed mutagenesis (see, .e.g., Carter et al., Nucl. Acids Res. 73:4331 (1986); and Zoller et al., Nucl. Acids Res. 70:6487 (1982)), cassette mutagenesis (see, e.g., Wells et al., Gene 34:315 (1985)), restriction selection mutagenesis (see, e.g., Wells et al., Philos. Trans. R. Soc. London SerA 377:415 (1986)).

Scanning amino acid analysis can also be employed to identify one or more amino acids along a contiguous sequence. Among the preferred scanning amino acids are relatively small neutral amino acids. Such amino acids include alanine, glycine, serine and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. If alanine substituting does not yield adequate amounts of variant, an isoteric amino acid can be used.

### EXAMPLES

Without further elaboration, it is believed that one skilled in the art, using the preceding description, can utilize the invention to the fullest extent. The following examples are illustrative only, and not limiting of the remainder of the disclosure in any way whatsoever.

### Example 1

### SERPINE2 is One of 144 Genes Differentially Expressed in Primary Colorectal Tumors

This example demonstrates SERPINE2 is one of 144 genes differentially expressed in primary colorectal tumors as shown by cDNA microarray analysis. See Table 1.

Forty-two CRC patients were identified and matched samples of primary colon tumor, adjacent normal colon epithelium, and, in most cases, metastatic liver tumors were obtained and flash frozen. Sample RNAs were obtained using laser capture microdissection and transcription-mediated amplification. Van Gelder et al., PROC. NATL. ACAD. SCI. USA 87(5): 1663-7 (1990); and Luo et al., NAT MED. 5(1): 117-22 (1999).

Gene expression was measured using custom two-color cDNA microarrays. Each sample was separately labeled with both cye-3 and cye-5 dyes prior to hybridization. Relative gene expression was measured between pairs of co-hybridized samples. As many as three measurements were observed: primary colon tumor relative to adjacent normal colon epithelium (T/N), metastatic liver tumor relative to adjacent normal epithelium (M/N), and metastatic liver tumor relative to primary colon tumor (M/T). All three of these measurements were made for a subset consisting of 32 patients. Each pair of samples was measured twice with alternating labeling to eliminate bias from the dyes. Each array was spotted in duplicate and the four resulting measurements were averaged. A signal model was used to correct for background, make detection calls for the signals, and assign uncertainty and statistical significance to the final gene expression ratios. For each sample where good T/N, M/T and M/N ratios were obtained (the mRNA expression was detected above background for both the numerator and denominator), consistent results for these three measurements were required such that the combined product should give a value of one within 25% (i.e., 4/5<(T/N)(M/T)/(M/N)<5/4.) Measurements not meeting this requirement were not used.

Genes were identified which were differentially expressed between metastatic liver tumors and primary colon tumors in at least 20% of the 32 colon patients where T/N, M/N and M/T expression ratios had all been measured. For a particular pair of metastatic and primary tumor samples, a gene was identified as over-expressed in the metastatic tumor if 1) the ratio of expression M/T was greater than 1.5, and 2) the 95% confidence limit associated with the ratio measurement was greater than one. Likewise, genes were identified as under-expressed in a pair of samples if 1) the ratio M/T was less than ⅔, and 2) the 95% confidence limit associated with the ratio measurement was less than one. Because the colon patients in the present study represent only a very small sample of the total colorectal cancer patient population, the following requirements were used to ensure that the genes identified as over- (or under-) expressed were very likely to be over- (or under-) expressed in at least 20% of the total population. At least 10 usable measurements were required for each spot. Also, given the number of positive observations (samples in which the gene was over- (or under-) expressed) and the total number of observations (samples with usable measurements), the lower 99% confidence limit on the probability of seeing over- (or under) expression in the total population was estimated from the binomial distribution using the method of maximum likelihood. Genes in which at least 20 percent of the measurements were differentially expressed to within a 99 percent confidence interval were identified.

Based on these criteria, 144 genes were identified that are differentially expressed between primary colon tumors and matched metastatic tumors to the liver in 20% of colorectal cancer patients. Interestingly, this analysis demonstrated that a very small fraction of genes measured were either up- or down-regulated in metastatic tumors relative the matched primary tumor. This may suggest that relatively few mutations are required for a primary tumor to acquire the propensity or ability to metastasize. The subset of the differentially expressed genes that consists of 68 genes that were identified as over-expressed in metastatic tumors relative to primary tumors is presented in Table 1. The subset that were identified as under-expressed is presented in Table 2. Hierarchical clustering of the genes using Pearson correlation as the distance metric was performed to identify groups of genes with similar expression profiles across patients. These genes may have similar functions or be co-regulated or otherwise interact with each other. The cluster to which each gene was assigned is listed in the tables.

TABLE 1
68 genes that show increased mRNA expression in hepatic metastases
relative to primary colon tumors from the same patient. Each gene is identified by its
LocusLink ID, symbol and name. The “SPOTS” column indicates the number of
spots (cDNA clones) used to measure each gene's expression. The “N” column
shows the maximum number of patient samples where a particular gene was
measured; this number was used in calculating the average percent prevalence across
patients. The average prevalence, for cuts of 1.5-, 2-, 4- and 10-fold increased M/T
expression, or cases where the gene was only detected in the metastatic tumor sample
(“ON”), are shown as a percentage of the patients where the gene was measured. The
“CLUST” column indicates which of the clusters, identified using hierarchical
clustering as described in the text, each gene belongs to. The genes are ordered by the
sum of the prevalence values, in decreasing order.

| LOCUSID | SYMBOL | GENE | SPOTS | N | >1.5X | >2X | >4X | >10X | ON | CLUST |
|---|---|---|---|---|---|---|---|---|---|---|
| 8991 | SELENBP1 | selenium binding protein 1 | 1 | 30 | 56.7% | 43.3% | 23.3% | 6.7% | 0.0% | 3 |
| 163732 | CITED4 | Cbp/p300-interacting | 1 | 26 | 53.8% | 38.5% | 23.1% | 7.7% | 0.0% | 3 |
| | | transactivator, with Glu/Asp-rich | | | | | | | | |
| | | carboxy-terminal domain, 4 | | | | | | | | |
| 4060 | LUM | lumican | 4 | 32 | 62.5% | 43.8% | 15.6% | 0.0% | 0.0% | 5 |
| 5266 | PI3 | protease inhibitor 3, skin-derived | 1 | 26 | 53.8% | 50.0% | 11.5% | 3.8% | 0.0% | 3 |
| | | (SKALP) | | | | | | | | |
| 27290 | SPINK4 | serine protease inhibitor, Kazal | 1 | 21 | 52.4% | 28.6% | 28.6% | 9.5% | 0.0% | 3 |
| | | type 4 | | | | | | | | |
| 27248 | XTP3TPB | XTP3-transactivated protein B | 1 | 26 | 50.0% | 34.6% | 19.2% | 7.7% | 0.0% | 3 |
| 9518 | GDF15 | growth differentiation factor 15 | 2 | 31 | 51.6% | 38.7% | 16.1% | 3.2% | 0.0% | 6 |
| 374354 | FLJ25621 | FLJ25621 protein | 1 | 23 | 52.2% | 34.8% | 17.4% | 4.3% | 0.0% | 3 |
| 2192 | FBLN1 | fibulin 1 | 2 | 15 | 53.3% | 46.7% | 6.7% | 0.0% | 0.0% | 5 |
| 11167 | FSTL1 | follistatin-like 1 | 2 | 31 | 61.3% | 41.9% | 3.2% | 0.0% | 0.0% | 5 |
| 4313 | MMP2 | matrix metalloproteinase 2 | 2 | 26 | 65.4% | 34.6% | 3.8% | 0.0% | 0.0% | 5 |
| | | (gelatinase A, 72 kDa gelatinase, | | | | | | | | |
| | | 72 kDa type IV collagenase) | | | | | | | | |
| 2049 | EPHB3 | EphB3 | 1 | 27 | 48.1% | 33.3% | 18.5% | 3.7% | 0.0% | 6 |
| 800 | CALD1 | caldesmon 1 | 1 | 28 | 50.0% | 39.3% | 10.7% | 3.6% | 0.0% | 5 |
| 57535 | KIAA1324 | maba1 | 1 | 27 | 51.9% | 33.3% | 7.4% | 3.7% | 3.7% | 3 |
| 4535 | MTND1 | NADH dehydrogenase 1 | 2 | 30 | 63.3% | 36.7% | 0.0% | 0.0% | 0.0% | 4 |
| 1634 | DCN | decorin | 6 | 32 | 53.1% | 28.1% | 15.6% | 0.0% | 0.0% | 5 |
| 26073 | POLDIP2 | polymerase (DNA-directed), delta | 1 | 27 | 51.9% | 29.6% | 14.8% | 0.0% | 0.0% | 3 |
| | | interacting protein 2 | | | | | | | | |
| 26047 | CNTNAP2 | contactin associated protein-like 2 | 1 | 21 | 52.4% | 28.6% | 4.8% | 0.0% | 9.5% | 4 |
| 1513 | CTSK | cathepsin K (pycnodysostosis) | 1 | 15 | 53.3% | 33.3% | 6.7% | 0.0% | 0.0% | 5 |
| 131177 | FAM3D | family with sequence similarity 3, | 1 | 29 | 48.3% | 24.1% | 13.8% | 6.9% | 0.0% | 3 |
| | | member D | | | | | | | | |
| 5786 | PTPRA | protein tyrosine phosphatase, | 1 | 29 | 58.6% | 34.5% | 0.0% | 0.0% | 0.0% | 4 |
| | | receptor type, A | | | | | | | | |
| 5327 | PLAT | plasminogen activator, tissue | 2 | 28 | 60.7% | 28.6% | 0.0% | 0.0% | 0.0% | 5 |
| 10406 | WFDC2 | WAP four-disulfide core domain 2 | 1 | 25 | 52.0% | 24.0% | 12.0% | 0.0% | 0.0% | 3 |
| 1278 | COL1A2 | collagen, type I, alpha 2 | 2 | 32 | 43.8% | 31.3% | 9.4% | 3.1% | 0.0% | 5 |
| 132160 | FLJ32332 | likely ortholog of mouse protein | 1 | 31 | 48.4% | 32.3% | 6.5% | 0.0% | 0.0% | 4 |
| | | phosphatase 2C eta | | | | | | | | |
| 4536 | MTND2 | NADH dehydrogenase 2 | 2 | 31 | 54.8% | 29.0% | 3.2% | 0.0% | 0.0% | 4 |
| 7433 | VIPR1 | vasoactive intestinal peptide | 1 | 21 | 57.1% | 28.6% | 0.0% | 0.0% | 0.0% | 3 |
| | | receptor 1 | | | | | | | | |
| 4091 | MADH6 | MAD, mothers against | 1 | 28 | 60.7% | 25.0% | 0.0% | 0.0% | 0.0% | 4 |
| | | decapentaplegic homolog 6 | | | | | | | | |
| | | (Drosophila) | | | | | | | | |
| 1277 | COL1A1 | collagen, type I, alpha 1 | 1 | 20 | 50.0% | 30.0% | 5.0% | 0.0% | 0.0% | 5 |
| 1504 | CTRB1 | chymotrypsinogen B1 | 1 | 30 | 53.3% | 26.7% | 3.3% | 0.0% | 0.0% | 4 |
| 6741 | SSB | Sjogren syndrome antigen B | 1 | 27 | 55.6% | 25.9% | 0.0% | 0.0% | 0.0% | 4 |
| | | (autoantigen La) | | | | | | | | |
| 27122 | DKK3 | dickkopf homolog 3 (Xenopus | 1 | 20 | 50.0% | 25.0% | 5.0% | 0.0% | 0.0% | 5 |
| | | laevis) | | | | | | | | |
| 23469 | PHF3 | PHD finger protein 3 | 1 | 30 | 60.0% | 20.0% | 0.0% | 0.0% | 0.0% | 4 |
| 5908 | RAP1B | RAP1B, member of RAS | 1 | 30 | 53.3% | 26.7% | 0.0% | 0.0% | 0.0% | 4 |
| | | oncogene family | | | | | | | | |
| 5295 | PIK3R1 | phosphoinositide-3-kinase, | 1 | 25 | 52.0% | 24.0% | 4.0% | 0.0% | 0.0% | 4 |
| | | regulatory subunit, polypeptide 1 | | | | | | | | |
| | | (p85 alpha) | | | | | | | | |
| 3297 | HSF1 | heat shock transcription factor 1 | 1 | 30 | 56.7% | 23.3% | 0.0% | 0.0% | 0.0% | 4 |
| 2 | A2M | alpha-2-macroglobulin | 1 | 30 | 46.7% | 26.7% | 6.7% | 0.0% | 0.0% | 5 |
| 81558 | LOC81558 | C/EBP-induced protein | 1 | 29 | 51.7% | 24.1% | 3.4% | 0.0% | 0.0% | 4 |
| 5801 | PTPRR | protein tyrosine phosphatase, | 1 | 29 | 44.8% | 31.0% | 3.4% | 0.0% | 0.0% | 3 |
| | | receptor type, R | | | | | | | | |
| 2487 | FRZB | frizzled-related protein | 1 | 18 | 55.6% | 16.7% | 5.6% | 0.0% | 0.0% | 3 |
| 6348 | CCL3 | chemokine (C-C motif) ligand 3 | 1 | 27 | 51.9% | 22.2% | 3.7% | 0.0% | 0.0% | 4 |
| 2006 | ELN | elastin (supravalvular aortic | 1 | 27 | 48.1% | 29.6% | 0.0% | 0.0% | 0.0% | 4 |
| | | stenosis, Williams-Beuren | | | | | | | | |
| | | syndrome) | | | | | | | | |
| 84859 | MGC4126 | hypothetical protein MGC4126 | 2 | 31 | 45.2% | 25.8% | 6.5% | 0.0% | 0.0% | 5 |
| 4513 | MTCO2 | cytochrome c oxidase II | 3 | 31 | 51.6% | 22.6% | 3.2% | 0.0% | 0.0% | 4 |
| 146880 | MGC40489 | hypothetical protein MGC40489 | 1 | 30 | 46.7% | 26.7% | 3.3% | 0.0% | 0.0% | 4 |
| 83483 | PLVAP | plasmalemma vesicle associated | 1 | 25 | 48.0% | 28.0% | 0.0% | 0.0% | 0.0% | 5 |
| | | protein | | | | | | | | |
| 6678 | SPARC | secreted protein, acidic, cysteine- | 1 | 29 | 44.8% | 24.1% | 6.9% | 0.0% | 0.0% | 5 |
| | | rich (osteonectin) | | | | | | | | |
| 132241 | LOC132241 | hypothetical protein LOC132241 | 1 | 29 | 51.7% | 20.7% | 3.4% | 0.0% | 0.0% | 4 |
| 67122 | Nrarp | Notch-regulated ankyrin repeat | 1 | 28 | 42.9% | 28.6% | 3.6% | 0.0% | 0.0% | 3 |
| | | protein | | | | | | | | |
| 398 | ARHGDIG | Rho GDP dissociation inhibitor | 1 | 27 | 44.4% | 18.5% | 11.1% | 0.0% | 0.0% | 4 |
| | | (GDI) gamma | | | | | | | | |
| 51599 | LISCH7 | liver-specific bHLH-Zip | 1 | 30 | 50.0% | 23.3% | 0.0% | 0.0% | 0.0% | 4 |
| | | transcription factor | | | | | | | | |
| 81788 | SNARK | likely ortholog of rat SNF1/AMP- | 1 | 29 | 51.7% | 20.7% | 0.0% | 0.0% | 0.0% | 4 |
| | | activated protein kinase | | | | | | | | |
| 57605 | PITPNM2 | phosphatidylinositol transfer | 1 | 29 | 51.7% | 20.7% | 0.0% | 0.0% | 0.0% | 4 |
| | | protein, membrane-associated 2 | | | | | | | | |
| 51332 | SPTBN5 | spectrin, beta, non-erythrocytic 5 | 1 | 25 | 48.0% | 20.0% | 4.0% | 0.0% | 0.0% | 4 |
| 4541 | MTND6 | NADH dehydrogenase 6 | 6 | 32 | 43.8% | 25.0% | 3.1% | 0.0% | 0.0% | 4 |
| 56940 | DUSP22 | dual specificity phosphatase 22 | 1 | 27 | 48.1% | 22.2% | 0.0% | 0.0% | 0.0% | 5 |
| 2580 | GAK | cyclin G associated kinase | 1 | 27 | 51.9% | 18.5% | 0.0% | 0.0% | 0.0% | 4 |
| 23432 | GPR161 | G protein-coupled receptor 161 | 1 | 29 | 48.3% | 20.7% | 0.0% | 0.0% | 0.0% | 4 |
| 583 | BBS2 | Bardet-Biedl syndrome 2 | 1 | 29 | 48.3% | 20.7% | 0.0% | 0.0% | 0.0% | 4 |
| 2045 | EPHA7 | EphA7 | 1 | 28 | 46.4% | 21.4% | 0.0% | 0.0% | 0.0% | 4 |
| 3189 | HNRPH3 | heterogeneous nuclear | 1 | 31 | 48.4% | 19.4% | 0.0% | 0.0% | 0.0% | 4 |
| | | ribonucleoprotein H3 (2H9) | | | | | | | | |
| 1158 | CKM | creatine kinase, muscle | 1 | 30 | 46.7% | 20.0% | 0.0% | 0.0% | 0.0% | 4 |
| 9775 | DDX48 | DEAD (Asp-Glu-Ala-Asp) box | 1 | 24 | 45.8% | 20.8% | 0.0% | 0.0% | 0.0% | 4 |
| | | polypeptide 48 | | | | | | | | |
| 10551 | AGR2 | anterior gradient 2 homolog | 2 | 29 | 41.4% | 24.1% | 0.0% | 0.0% | 0.0% | 3 |
| | | (Xenopus laevis) | | | | | | | | |
| 1999 | ELF3 | E74-like factor 3 (ets domain | 1 | 30 | 43.3% | 20.0% | 0.0% | 0.0% | 0.0% | 3 |
| | | transcription factor, epithelial- | | | | | | | | |
| | | specific) | | | | | | | | |
| 27336 | HTATSF1 | HIV TAT specific factor 1 | 1 | 29 | 44.8% | 17.2% | 0.0% | 0.0% | 0.0% | 4 |
| 7125 | TNNC2 | troponin C2, fast | 1 | 28 | 42.9% | 14.3% | 0.0% | 0.0% | 0.0% | 4 |
| 3040 | HBA2 | hemoglobin, alpha 2 | 1 | 27 | 48.1% | 3.7% | 0.0% | 0.0% | 0.0% | 5 |

TABLE 2
76 genes that show decreased mRNA expression in hepatic metastases
relative to primary colon tumors from the same patient. The columns are the same as
in Table 1, except the prevalence columns indicate percentage of patients exhibiting
down regulation, and the “OFF” column indicates the percentage of patients where
expression was detected in the primary tumor, but not the metastasis.

| LOCUSID | SYMBOL | GENE | SPOTS | N | >1.5X | >2X | >4X | >10X | ON | CLUST |
|---|---|---|---|---|---|---|---|---|---|---|
| 12 | SERPINA3 | serine (or cysteine) proteinase | 2 | 25 | 64.0% | 60.0% | 48.0% | 24.0% | 0.0% | 1 |
| | | inhibitor, clade A (alpha-1 | | | | | | | | |
| | | antiproteinase, antitrypsin), member 3 | | | | | | | | |
| 51761 | ATP8A2 | ATPase, aminophospholipid | 1 | 15 | 60.0% | 53.3% | 40.0% | 26.7% | 0.0% | 1 |
| | | transporter-like, Class I, type 8A, | | | | | | | | |
| | | member 2 | | | | | | | | |
| 51129 | ANGPTL4 | angiopoietin-like 4 | 1 | 18 | 66.7% | 61.1% | 22.2% | 5.6% | 0.0% | 2 |
| 348 | APOE | apolipoprotein E | 2 | 32 | 59.4% | 50.0% | 31.3% | 6.3% | 0.0% | 5 |
| 5004 | ORM1 | orosomucoid 1 | 1 | 19 | 57.9% | 42.1% | 26.3% | 15.8% | 0.0% | 1 |
| 3240 | HP | haptoglobin | 7 | 29 | 51.7% | 41.4% | 27.6% | 17.2% | 0.0% | 1 |
| 6347 | CCL2 | chemokine (C-C motif) ligand 2 | 2 | 21 | 71.4% | 42.9% | 14.3% | 4.8% | 0.0% | 5 |
| 27295 | PDLIM3 | PDZ and LIM domain 3 | 1 | 14 | 64.3% | 50.0% | 7.1% | 0.0% | 7.1% | 5 |
| 213 | ALB | albumin | 1 | 14 | 50.0% | 35.7% | 21.4% | 14.3% | 0.0% | 2 |
| 7076 | TIMP1 | tissue inhibitor of metalloproteinase 1 | 3 | 31 | 67.7% | 41.9% | 6.5% | 0.0% | 0.0% | 6 |
| | | (erythroid potentiating activity, | | | | | | | | |
| | | collagenase inhibitor) | | | | | | | | |
| 2335 | FN1 | fibronectin 1 | 4 | 32 | 56.3% | 40.6% | 18.8% | 0.0% | 0.0% | 6 |
| 710 | SERPING1 | serine (or cysteine) proteinase | 1 | 26 | 50.0% | 34.6% | 26.9% | 3.8% | 0.0% | 5 |
| | | inhibitor, clade G (C1 inhibitor), | | | | | | | | |
| | | member 1, (angioedema, hereditary) | | | | | | | | |
| 8553 | BHLHB2 | basic helix-loop-helix domain | 1 | 26 | 57.7% | 53.8% | 0.0% | 0.0% | 0.0% | 6 |
| | | containing, class B, 2 | | | | | | | | |
| 3303 | HSPA1A | heat shock 70 kDa protein 1A | 4 | 31 | 48.4% | 35.5% | 22.6% | 3.2% | 0.0% | 3 |
| 6373 | CXCL11 | chemokine (C—X—C motif) ligand 11 | 1 | 11 | 63.6% | 18.2% | 9.1% | 0.0% | 18.2% | 5 |
| 56937 | TMEPAI | transmembrane, prostate androgen | 1 | 25 | 48.0% | 40.0% | 16.0% | 4.0% | 0.0% | 6 |
| | | induced RNA | | | | | | | | |
| 6035 | RNASE1 | ribonuclease, RNase A family, 1 | 1 | 29 | 55.2% | 31.0% | 13.8% | 6.9% | 0.0% | 3 |
| | | (pancreatic) | | | | | | | | |
| 2316 | FLNA | filamin A, alpha (actin binding protein | 2 | 31 | 51.6% | 41.9% | 12.9% | 0.0% | 0.0% | 5 |
| | | 280) | | | | | | | | |
| 768 | CA9 | carbonic anhydrase IX | 1 | 18 | 44.4% | 33.3% | 16.7% | 0.0% | 11.1% | 6 |
| 2885 | GRB2 | growth factor receptor-bound protein 2 | 1 | 21 | 57.1% | 33.3% | 14.3% | 0.0% | 0.0% | 5 |
| 7564 | ZNF16 | zinc finger protein 16 (KOX 9) | 1 | 27 | 51.9% | 44.4% | 7.4% | 0.0% | 0.0% | 6 |
| 4070 | TACSTD2 | tumor-associated calcium signal | 1 | 28 | 50.0% | 28.6% | 17.9% | 7.1% | 0.0% | 6 |
| | | transducer 2 | | | | | | | | |
| 7052 | TGM2 | transglutaminase 2 (C polypeptide, | 1 | 28 | 57.1% | 32.1% | 14.3% | 0.0% | 0.0% | 6 |
| | | protein-glutamine-gamma- | | | | | | | | |
| | | glutamyltransferase) | | | | | | | | |
| 4071 | TM4SF1 | transmembrane 4 superfamily | 1 | 29 | 51.7% | 41.4% | 6.9% | 3.4% | 0.0% | 4 |
| | | member 1 | | | | | | | | |
| 10397 | NDRG1 | N-myc downstream regulated gene 1 | 1 | 30 | 56.7% | 40.0% | 6.7% | 0.0% | 0.0% | 2 |
| 2207 | FCER1G | Fc fragment of IgE, high affinity I, | 1 | 21 | 61.9% | 33.3% | 4.8% | 0.0% | 0.0% | 5 |
| | | receptor for; gamma polypeptide | | | | | | | | |
| 1152 | CKB | creatine kinase, brain | 3 | 32 | 50.0% | 31.3% | 12.5% | 6.3% | 0.0% | 3 |
| 1514 | CTSL | cathepsin L | 4 | 31 | 54.8% | 29.0% | 9.7% | 3.2% | 0.0% | 5 |
| 652 | BMP4 | bone morphogenetic protein 4 | 1 | 24 | 50.0% | 37.5% | 8.3% | 0.0% | 0.0% | 6 |
| 633 | BGN | biglycan | 1 | 24 | 58.3% | 37.5% | 0.0% | 0.0% | 0.0% | 1 |
| 3936 | LCP1 | lymphocyte cytosolic protein 1 (L- | 1 | 23 | 47.8% | 26.1% | 13.0% | 8.7% | 0.0% | 5 |
| | | plastin) | | | | | | | | |
| 2878 | GPX3 | glutathione peroxidase 3 (plasma) | 1 | 18 | 55.6% | 33.3% | 5.6% | 0.0% | 0.0% | 1 |
| 7422 | VEGF | vascular endothelial growth factor | 2 | 30 | 53.3% | 33.3% | 6.7% | 0.0% | 0.0% | 2 |
| 4256 | MGP | matrix Gla protein | 1 | 30 | 46.7% | 36.7% | 10.0% | 0.0% | 0.0% | 1 |
| 374 | AREG | amphiregulin (schwannoma-derived | 1 | 29 | 44.8% | 27.6% | 13.8% | 6.9% | 0.0% | 4 |
| | | growth factor) | | | | | | | | |
| 7701 | ZNF142 | zinc finger protein 142 (clone pHZ- | 1 | 28 | 46.4% | 32.1% | 14.3% | 0.0% | 0.0% | 3 |
| | | 49) | | | | | | | | |
| 51366 | DD5 | progestin induced protein | 1 | 27 | 44.4% | 37.0% | 11.1% | 0.0% | 0.0% | 6 |
| 10457 | GPNMB | glycoprotein (transmembrane) nmb | 1 | 26 | 50.0% | 30.8% | 11.5% | 0.0% | 0.0% | 5 |
| 22822 | PHLDA1 | pleckstr in homology-like domain, | 1 | 24 | 50.0% | 33.3% | 8.3% | 0.0% | 0.0% | 6 |
| | | family A, member 1 | | | | | | | | |
| 10123 | ARL7 | ADP-ribosylation factor-like 7 | 3 | 32 | 46.9% | 31.3% | 6.3% | 6.3% | 0.0% | 6 |
| 4316 | MMP7 | matrix metalloproteinase 7 | 6 | 32 | 46.9% | 31.3% | 9.4% | 3.1% | 0.0% | 6 |
| | | (matrilysin, uterine) | | | | | | | | |
| 3576 | IL8 | interleukin 8 | 1 | 21 | 47.6% | 33.3% | 4.8% | 4.8% | 0.0% | 6 |
| 5163 | PDK1 | pyruvate dehydrogenase kinase, | 1 | 29 | 58.6% | 31.0% | 0.0% | 0.0% | 0.0% | 2 |
| | | isoenzyme 1 | | | | | | | | |
| 1649 | DDIT3 | DNA-damage-inducible transcript 3 | 1 | 28 | 46.4% | 35.7% | 7.1% | 0.0% | 0.0% | 2 |
| 7033 | TFF3 | trefoil factor 3 (intestinal) | 1 | 27 | 44.4% | 22.2% | 14.8% | 3.7% | 3.7% | 3 |
| 4493 | MT1E | metallothionein 1E (functional) | 1 | 26 | 46.2% | 30.8% | 11.5% | 0.0% | 0.0% | 3 |
| 7481 | WNT11 | wingless-type MMTV integration site | 1 | 17 | 52.9% | 23.5% | 5.9% | 0.0% | 5.9% | 4 |
| | | family, member 11 | | | | | | | | |
| 10381 | TUBB4 | tubulin, beta, 4 | 1 | 15 | 60.0% | 26.7% | 0.0% | 0.0% | 0.0% | 1 |
| 5270 | SERPINE2 | serine (or cysteine) proteinase | 1 | 28 | 46.4% | 32.1% | 3.6% | 3.6% | 0.0% | 6 |
| | | inhibitor, clade E (nexin, plasminogen | | | | | | | | |
| | | activator inhibitor type 1), member 2 | | | | | | | | |
| 9590 | AKAP12 | A kinase (PRKA) anchor protein | 1 | 20 | 50.0% | 25.0% | 5.0% | 5.0% | 0.0% | 2 |
| | | (gravin) 12 | | | | | | | | |
| 54206 | MIG-6 | mitogen-inducible gene 6 | 1 | 31 | 45.2% | 35.5% | 3.2% | 0.0% | 0.0% | 2 |
| 11067 | C10orf10 | chromosome 10 open reading frame | 1 | 24 | 62.5% | 20.8% | 0.0% | 0.0% | 0.0% | 6 |
| | | 10 | | | | | | | | |
| 2159 | F10 | coagulation factor X | 1 | 18 | 50.0% | 33.3% | 0.0% | 0.0% | 0.0% | 4 |
| 57561 | ARRDC3 | arrestin domain containing 3 | 3 | 27 | 59.3% | 22.2% | 0.0% | 0.0% | 0.0% | 6 |
| 4495 | MT1G | metallothionein 1G | 1 | 27 | 44.4% | 22.2% | 11.1% | 3.7% | 0.0% | 3 |
| 10628 | TXNIP | thioredoxin interacting protein | 5 | 32 | 46.9% | 31.3% | 3.1% | 0.0% | 0.0% | 3 |
| 3939 | LDHA | lactate dehydrogenase A | 2 | 32 | 53.1% | 25.0% | 3.1% | 0.0% | 0.0% | 6 |
| 6890 | TAP1 | transporter 1, ATP-binding cassette, | 1 | 24 | 45.8% | 20.8% | 8.3% | 4.2% | 0.0% | 3 |
| | | sub-family B (MDR/TAP) | | | | | | | | |
| 1942 | EFNA1 | ephrin-A1 | 1 | 28 | 46.4% | 32.1% | 0.0% | 0.0% | 0.0% | 4 |
| 136 | ADORA2B | adenosine A2b receptor | 1 | 22 | 50.0% | 27.3% | 0.0% | 0.0% | 0.0% | 3 |
| 11145 | HRASLS3 | HRAS-like suppressor 3 | 1 | 26 | 46.2% | 30.8% | 0.0% | 0.0% | 0.0% | 6 |
| 84951 | CTEN | C-terminal tensin-like | 1 | 21 | 52.4% | 19.0% | 4.8% | 0.0% | 0.0% | 6 |
| 90637 | LOC90637 | hypothetical protein LOC90637 | 1 | 29 | 44.8% | 27.6% | 3.4% | 0.0% | 0.0% | 2 |
| 7045 | TGFBI | transforming growth factor, beta- | 1 | 24 | 50.0% | 25.0% | 0.0% | 0.0% | 0.0% | 6 |
| | | induced, 68 kDa | | | | | | | | |
| 5230 | PGK1 | phosphoglycerate kinase 1 | 1 | 32 | 46.9% | 25.0% | 3.1% | 0.0% | 0.0% | 6 |
| 64065 | PERP | PERP, TP53 apoptosis effector | 1 | 28 | 46.4% | 17.9% | 3.6% | 3.6% | 0.0% | 6 |
| 9531 | BAG3 | BCL2-associated athanogene 3 | 1 | 28 | 42.9% | 25.0% | 3.6% | 0.0% | 0.0% | 2 |
| 23023 | KIAA0779 | KIAA0779 protein | 1 | 21 | 47.6% | 23.8% | 0.0% | 0.0% | 0.0% | 3 |
| 567 | B2M | beta-2-microglobulin | 1 | 28 | 46.4% | 17.9% | 0.0% | 0.0% | 0.0% | 3 |
| 2317 | FLNB | filamin B, beta (actin binding protein | 1 | 28 | 42.9% | 14.3% | 7.1% | 0.0% | 0.0% | 3 |
| | | 278) | | | | | | | | |
| 665 | BNIP3L | BCL2/adenovirus E1B 19 kDa | 2 | 30 | 46.7% | 16.7% | 0.0% | 0.0% | 0.0% | 2 |
| | | interacting protein 3-like | | | | | | | | |
| 7431 | VIM | vimentin | 1 | 28 | 42.9% | 14.3% | 3.6% | 0.0% | 0.0% | 5 |
| 91452 | ACBD5 | acyl-Coenzyme A binding domain | 1 | 27 | 44.4% | 14.8% | 0.0% | 0.0% | 0.0% | 4 |
| | | containing 5 | | | | | | | | |
| 1508 | CTSB | cathepsin B | 1 | 31 | 48.4% | 9.7% | 0.0% | 0.0% | 0.0% | 6 |
| 55818 | JMJD1 | jumonji domain containing 1 | 1 | 28 | 42.9% | 14.3% | 0.0% | 0.0% | 0.0% | 6 |
| 5763 | PTMS | parathymosin | 1 | 31 | 41.9% | 12.9% | 0.0% | 0.0% | 0.0% | 3 |

### Example 2

### Several Genes Differentially Expressed in Primary Colorectal Tumors Directly Interact with One Another

This example demonstrates that many of the genes identified in Table 1 as being expressed differentially in primary colorectal tumors directly interact with one another.

A proprietary software package, PathwayAssist™ version 2.52 (Ariadne Genomics Inc., Rockville, Md.), was used to identify and visualize biological associations between genes and gene products identified in Table 1. These associations were extracted by Ariadne Genomics Inc. using natural language processing techniques from article abstracts made freely available by the National Library of Medicine. The software was used to build biological associate networks including such relationships as protein binding, gene regulation, and phosphorylation. This database was searched to determine whether any direct interactions between these genes or gene products were identified in the scientific literature. Relationships were found between 37 of these genes or gene products.

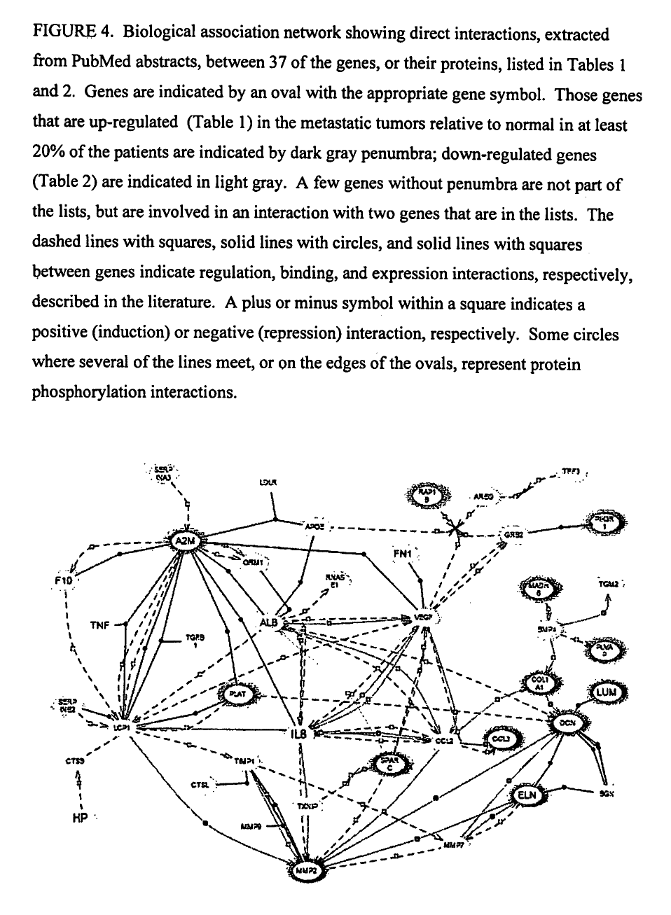

Referring to FIG. 4, a chart was generated to visualize the relationships between the above 37 genes or gene products. Importantly, several of these 37 genes or gene products have been identified as having important roles in cancer progression or metastasis. These include cathepsin B (CTSB), larval cuticular protein one (LCP1), matrix metalloproteinase two (MMP2), matrix metalloproteinase seven (MMP7), plasminogen activator (PLAT), serine proteinase inhibitor, clade E, member 2 (SERPINE2), tissue inhibitor of matrix metalloproteinases 1 (TIMP1), and vascular endothelial growth factor (VEGF).

It is important to identify and visualize the biological interaction of differentially expressed genes. The identified interactions are often called biological association networks. These networks assist in the overall understanding of disease mechanisms and may reflect a wide variety of interactions, including protein binding partners, regulation of gene expression, or regulation of protein modification. In some cases, it may be possible to identify, utilizing a biological association network and experimental data demonstrating differential expression, genes which are important to the progression and metastasis of cancer. In this example, 37 genes or gene products, that were previously identified as differentially expressed in Table 1, were also identified as interacting with one another.

### Example 3

### Differential Expression of SERPINE2 in Primary Colorectal Tumors is Linearly Correlated to Patient Survival

This example demonstrates that SERPINE2 expression in primary colorectal tumors is a negative prognostic indicator of CRC patient survival.

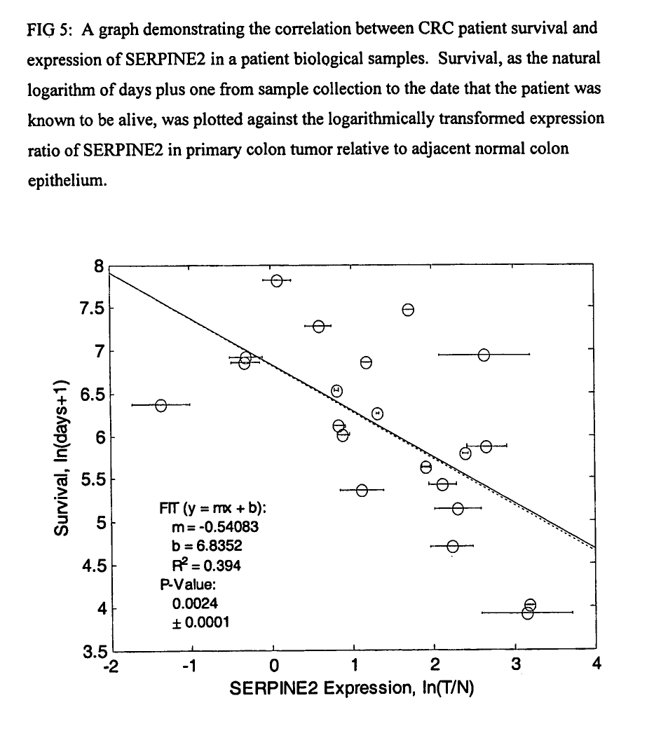

Parallel to the collection of treatment and control biological samples, clinical data were collected from each of the 42 CRC patients with informed consent. Data included patient outcomes and treatment details, such as clinical response, disease progression, duration from detection of primary tumor to detection of metastatic disease. Referring to FIG. 5, linear regression analysis was performed against each of these variables and final patient outcome demonstrated a strong negative correlation (p-value <0.001) between patient survival and expression of SERPINE2 in primary colon tumor relative to adjacent normal colon epithelium. T/N ratios measured in samples from 21 patients were used; in the remaining patients' samples, expression of SERPINE2 was not detected in either the primary tumor or adjacent normal sample, making the ratios unreliable. In calculating this regression, the gene expression ratio was logarithmically transformed and survival was represented as the natural log of the number of days, plus one, from sample collection to the last known date the patient was alive. Robustness of the linear regression was assessed using the bootstrap technique, and significance was assessed by an incomplete Fisher permutation test. The reported p-value is the most conservative calculated by these techniques. In each case, the influence of uncertainty in gene expression ratios were assessed by replacing each log ratio with a random value taken from a normal distribution defined by the mean log ratio and an appropriate standard deviation. As can be seen by FIG. 3, there is a strong negative correlation of CRC patient survival with the level of expression of SERPINE2 in primary colon tumors.

### Example 5

### Quantitative RT-PCR Results Validate the Microarray Results for SERPINE2

Two-step quantitative RT-PCR was used to validate the microarray results for SERPINE2 in additional patient samples. SERPINE2 expression was measured by TaqMan assays (Applied Biosystems, Foster City, Calif.) using amplified RNA samples of primary colon tumors and matched adjacent normal epithelium, obtained by LCM, from a total of 41 patients, including all but one of those measured on the cDNA microarrays (there was insufficient material from the remaining patient). GAPD (assay ID Hs99999905_ml, RefSeq NM_{—}002046) and SERPINE2 (assay ID Hs00299953_ml, RefSeq NM_{—}006216) PCR primers and reporter probes were obtained from Assays-on-Demand Gene Expression Kits (Applied Biosystems, Foster City, Calif.); GAPD was used for normalization between samples. The PCR reaction was done according to the Assays-on-Demand protocol for a 25 μL reaction, with 2 μL (10% of the reverse-transcription reaction product) from the cDNA preparation used as the template for each PCR reaction. The reaction parameters were as follows: 10 minute denaturation at 95 C, followed by 15 seconds at 95 C and 1 minute at 60 C for 45 cycles. The assays were run in a 96-well plate format, and a separate calibration based on a serial dilution of standards (first-strand cDNA from MDA-MB-435 cell line RNA) was performed for each plate. The quantities of SERPINE2 and GAPD were measured in each tissue sample at least three times, on separate days. The SERPINE2 RNA levels measured by cDNA microarrays and TaqMan are in good overall agreement with each other with an R² of 0.78 (excluding a single outlier).

### Example 6

### Patients with Higher Levels of SERPINE2 Over-Expression in Their Primary Colon Tumors Relative to Nearby Normal Colon Epithelium Have a Significantly Shorter Median Survival Time

This example further demonstrates that over-expression of SERPINE2 is a negative prognostic indicator of CRC patient survival.

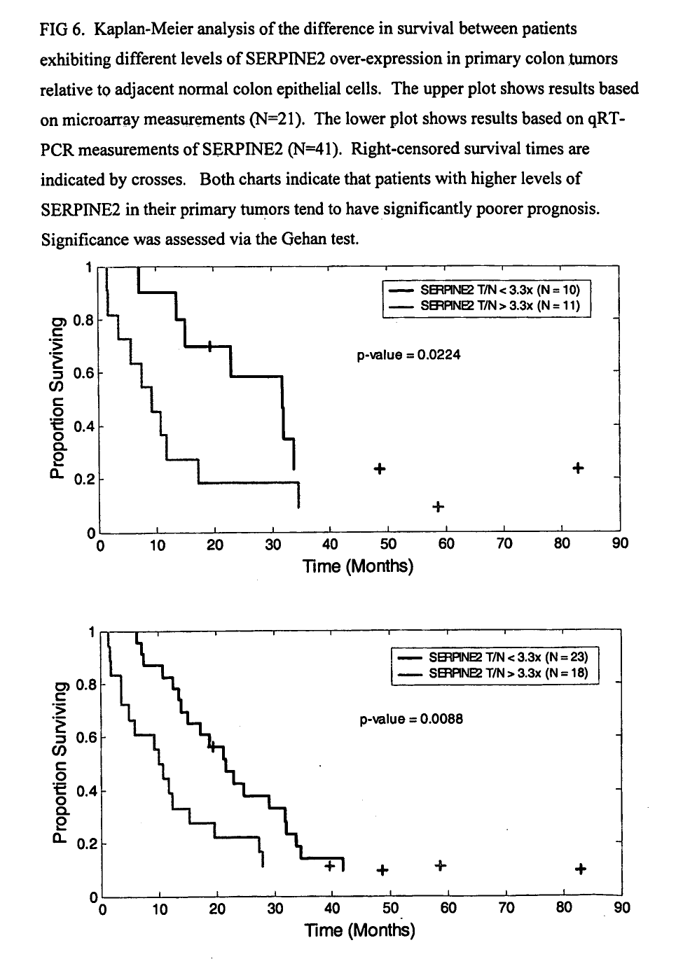

A Kaplan-Meier analysis of survival was performed; the results are illustrated in FIG. 6. In FIG. 6a, data derived from microarray measurements are illustrated. Note that not all of the patients who had matched N, T and M samples were used in this analysis, because their SERPINE2 expression level may have been below the microarray detection limit or otherwise failed to pass the quality criteria. In FIG. 6b, data derived from qRT-PCR measurements of primary tumor and normal tissue samples from 41 patients, including 20 of the 21 patients measured using the arrays, is illustrated. In each of the graphs, patients were divided into two groups of nearly equal size based on their level of SERPINE2 T/N expression, with T/N=3.3 as the cut. For each group, the fraction of patients surviving as a function of time is plotted. When applied to the microarray data, the Gehan test indicates a significant difference in survival between the two groups, with a p-value of 0.02. Although the difference in median survival shown in FIG. 6b is not as great among all 41 patients, the larger number of patients gives this latter result greater statistical significance, with a Gehan p-value 0.009.

1. A method for predicting the course of malignant disease in an individual, comprising
(a) determining levels of expression of SERPINE2 within a patient biological sample and a control biological sample;
(b) comparing the results of step (a); and
(c) correlating the comparison of step (b) to a predicted course of said malignant disease.

2. The method of claim 1, wherein the malignant disease is colorectal carcinoma.

3. The method of claim 1, wherein the malignant disease is hepatocellular carcinoma.

4. The method of claim 1, wherein the malignant disease is pancreatic carcinoma.

5. The method of claim 1, wherein the malignant disease is adult soft tissue sarcoma.

6. The method of claim 1, wherein the malignant disease is a carcinoma except for pancreatic carcinoma.

7. The method of claim 1, wherein the patient biological sample is malignant tissue obtained from said individual.

8. The method of claim 1, wherein the control biological sample is a predetermined standard.

9. The method of claim 1, wherein the control biological sample is an immortalized cell tissue culture.

10. The method of claim 1, wherein the control biological sample is a primary cell tissue culture.

11. The method of claim 1, wherein the control biological sample is non-malignant tissue of the same type as the patient biological sample.

12. The method of claim 1, wherein the patient biological sample is tissue from a metastatic tumor and the control biological sample is tissue from a primary tumor.

13. The method of claim 11, wherein both the patient and control biological samples are obtained from the same individual.

14. The method of claim 12, wherein both the patient and control biological samples are obtained from the same individual.

15. The method of claim 1, wherein the level of expression of SERPINE2 is determined utilizing mRNA.

16. The method of claim 1, wherein the level of expression of SERPINE2 is determined utilizing cDNA.

17. The method of claim 1, wherein the level of expression of SERPINE2 is determined utilizing SERPINE2 protein.

18. The method of claim 1, wherein the level of expression of SERPINE2 is determined utilizing labeled nucleic acids.

19. The method of claim 18, wherein said label is selected from the group consisting of a radiolabel, an enzyme, a chromophore, and a fluorophore.

20. The method of claim 1, wherein the level of expression of SERPINE2 is determined using either northern blot hybridization or a fixed-array of mRNA targets.

21. The method of claim 1, wherein the level of expression of SERPINE2 is determined using either southern blot hybridization or a fixed array of cDNA targets.

22. The method of claim 1, wherein the level of expression of SERPINE2 is determined using either western blot or a fixed array of protein targets.

23. The method of claim 1, wherein the level of expression of SERPINE2 is determined utilizing anti-SERPINE2 antibodies.

24. The method of claim 23, wherein said antibodies are labeled.

25. The method of claim 24, wherein said label is selected from the group consisting of a radiolabel, an enzyme, a chromophore, and a fluorophore.

26. A method for predicting a relapse of a malignant disease in an individual, comprising
(a) determining levels of expression of SERPINE2 within a patient biological sample and a control biological sample;
(b) comparing the results of step (a); and
(c) correlating the comparison of step (b) to a predicted course of said malignant disease.

27. The method of claim 26, wherein the malignant disease is colorectal carcinoma.

28. The method of claim 26, wherein the malignant disease is hepatocellular carcinoma.

29. The method of claim 26, wherein the malignant disease is pancreatic carcinoma.

30. The method of claim 26, wherein the malignant disease is adult soft tissue sarcoma.

31. The method of claim 26, wherein the malignant disease is a carcinoma except for pancreatic carcinoma.

32. The method of claim 26, wherein the patient biological sample is malignant tissue obtained from said individual.

33. The method of claim 26, wherein the control biological sample is a predetermined standard.

34. The method of claim 26, wherein the control biological sample is an immortalized cell tissue culture.

35. The method of claim 26, wherein the control biological sample is a primary cell tissue culture.

36. The method of claim 26, wherein the control biological sample is non-malignant tissue of the same type as the patient biological sample.

37. The method of claim 26, wherein the patient biological sample is tissue from a metastatic tumor and the control biological sample is tissue from a primary tumor.

38. The method of claim 36, wherein both the patient and control biological samples are obtained from the same individual.

39. The method of claim 37, wherein both the patient and control biological samples are obtained from the same individual.

40. The method of claim 26, wherein the level of expression of SERPINE2 is determined utilizing mRNA.

41. The method of claim 26, wherein the level of expression of SERPINE2 is determined utilizing cDNA.

42. The method of claim 26, wherein the level of expression of SERPINE2 is determined utilizing SERPINE2 protein.

43. The method of claim 26, wherein the level of expression of SERPINE2 is determined utilizing labeled nucleic acids.

44. The method of claim 43, wherein the label is selected from the group consisting of a radiolabel, an enzyme, a chromophore, and a fluorophore.

45. The method of claim 26, wherein the level of expression of SERPINE2 is determined using either northern blot hybridization or a fixed-array of mRNA targets.

46. The method of claim 26, wherein the level of expression of SERPINE2 is determined using either southern blot hybridization or a fixed array of cDNA targets.

47. The method of claim 26, wherein the level of expression of SERPINE2 is determined using either western blot or a fixed array of protein targets.

48. The method of claim 26, wherein the level of expression of SERPINE2 is determined utilizing anti-SERPINE2 antibodies.

49. The method of claim 48, wherein said antibodies are labeled.

50. The method of claim 49, wherein said label is selected from the group consisting of a radiolabel, an enzyme, a chromophore, and a fluorophore.

51. The method of claim 1, further comprising the correlation of SERPINE2 expression with additional prognostic indicators.

52. A kit useful for the prognosis of a malignant disease, comprising a fixed array of nucleotide or protein targets wherein at least one target specifically detects SERPINE2.

53. The kit of claim 52, wherein at least one target is at least 12 contiguous nucleotides substantially identical to either the nucleotide sequence in FIG. 1 or the nucleotide sequence in FIG. 2.

54. The kit of claim 52, wherein the protein target is a protein capture agent.

55. The kit of claim 53, wherein the protein capture agent is either a protein with an amino acid sequence set forth in FIG. 3 or a protein with an amino acid sequence consisting of at least 12 consecutive amino acids in FIG. 3.

56. The kit of claim 53, wherein said protein capture agent is an antibody or functional equivalent thereof that binds SERPINE2.

57. The kit of claim 53, wherein said protein capture agent is a SERPINE2 binding partner.

58. The kit of claim 52, further comprising at least one target specifically detecting another gene or gene product useful as a prognostic indicator.

59. A method for detecting gene or gene products useful as prognostic indicators of a malignant disease, comprising
(a) determining levels of expression of SERPINE2 within a patient biological sample and a control biological sample;
(b) determining levels of expression of another gene or gene product within a patient biological sample and a control biological sample
(c) comparing the results of step (a) and step (b); and
(d) correlating the comparison of step (c) to predict the usefulness of the gene or gene product as a prognostic indicator of a malignant disease.

60. The method of claim 26, further comprising the correlation of SERPINE2 expression with additional prognostic indicators.


SEQUENCE LISTING (3 sequences)

SEQ ID NO: 1 - DNA, 2129 bp, Homo sapiens
ggcacgagggctgcccgggctgcgcggcgtctgcaggcgccaccgctgcctctttccggctgtgaccctcctcgccggcgccgcttggctgcgtcctccgactccccgcgccgccgagaccaggctcccgctccggttgcggccgcaccgccctccgcggccgccccctggggatccagcgagcgcggtcgtccttggtggaaggaaccatgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaatgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtgcccaagagggtgcaggtgatcctgcccaagttcacagctgtagcacaaacagatttgaaggagccgctgaaagttcttggcattactgacatgtttgattcatcaaaggcaaattttgcaaaaataacaacagggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccctgaagagtatacaaaagaaaccatgcaaagcaacgactactttgctacgaagaaagactcctttcctgcatctttcatagttctgttaaatatttttgtacatcgcttctttttcaaaactagttcttaggaacagactcgatgcaagtgtttctgttctgggaggtattggagggaaaaaacagcaggatggctggaacactgtactgaggaatgaatagaaaggcttccagatgtctaaaagattctttaaactactgaactgttacctaggttaacaaccctgttgagtatttgctgtttgtccagttcaggaatttttgttttgttttgtctatatgtgcggcttttcagaagaaatttaatcagtgtgacagaaaaaaaaaatgttttatggtagcttttactttttatgaaaaaaaaattatttgccttttaaattcttttcccccatccccctccaaagtcttgatagcaagcgttattttgggggtagaaacggtgaaatctctagcctctttgtgtttttgttgttgttgttgttgttgttttatataatgcatgtattcactaaaataaaatttaaaaaactcctgtcttgctagacaaggttgctgttgtgcagtgtgcctgtcactactggtctgtactccttggatttgcatttttgtattttgtacaaagtaaaaataaactgttatgagtagtaaaaaaaaaaaaaaaaaa

SEQ ID NO: 2 - DNA, 2120 bp, Homo sapiens
gctgcccgggctgcgcggcgtctgcaggcgccaccgctgcctctttccggctgtgaccctcctcgccggcgccgcttggctgcgtcctccgactccccgcgccgccgagaccaggctcccgctccggttgcggccgcaccgccctccgcggccgccccctggggatccagcgagcgcggtcgtccttggtggaaggaaccatgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaatgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtgcccaagagggtgcaggtgatcctgcccaagttcacagctgtagcacaaacagatttgaaggagccgctgaaagttcttggcattactgacatgtttgattcatcaaaggcaaattttgcaaaaataacaacagggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccctgaagagtatacaaaagaaaccatgcaaagcaacgactactttgctacgaagaaagactcctttcctgcatctttcatagttctgttaaatatttttgtacatcgcttctttttcaaaactagttcttaggaacagactcgatgcaagtgtttctgttctgggaggtattggagggaaaaaacagcaggatggctggaacactgtactgaggaatgaatagaaaggcttccagatgtctaaaagattctttaaactactgaactgttacctaggttaacaaccctgttgagtatttgctgtttgtccagttcaggaatttttgttttgttttgtctatatgtgcggcttttcagaagaaatttaatcagtgtgacagaaaaaaaaaatgttttatggtagcttttactttttatgaaaaaaaaattatttgccttttaaattcttttcccccatccccctccaaagtcttgatagcaagcgttattttgggggtagaaacggtgaaatctctagcctctttgtgtttttgttgttgttgttgttgttgttttatataatgcatgtattcactaaaataaaatttaaaaaactcctgtcttgctagacaaggttgctgttgtgcagtgtgcctgtcactactggtctgtactccttggatttgcatttttgtattttgtacaaagtaaaaataaactgttatgagtagtaaaaaaaaaaaaaaaaaa

SEQ ID NO: 3 - protein, 398 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITTGSENLHVSHILQKAKIEVSEDGTKASAATTAILIARSSPPWFIVDRPFLFFIRHNPTGAVLFMGQINKP